(19) United States
(12) Patent Application Publication
(10) Pub. No.: US 2024/0252658 A1
(43) Pub. Date: Aug. 1, 2024

(54) BIFUNCTIONAL PROTAC-TYPE COMPOUNDS TARGETING PXR, METHOD FOR PREPARING SAME AND THERAPEUTIC USE THEREOF

(71) Applicants: Centre National de la Recherche Scientifique, PARIS (FR); Université de Montpellier, MONTPELLIER (FR); Institut National de la Santé et de la Recherche Médicale, PARIS (FR); École Nationale Supérieure de Chimie de Montpellier, MONTPELLIER (FR)

(72) Inventors: Jean-Marc PASCUSSI, VERGEZE (FR); Julie PANNEQUIN, SÈTE (FR); Muriel AMBLARD-CAUSSIL, SAINT VINCENT DE BARBEYRARGUES (FR); Guillaume LACONDE, MONTPELLIER (FR); Sabine GERBAL-CHALOIN, PRADES-LE-LEZ (FR); Alain CHAVANIEU, ASSAS (FR); William BOURGUET, SAINT GELY DU FESC (FR); Vanessa DELFOSSE, PRADES-LE-LEZ (FR)

(21) Appl. No.: 18/562,251

(22) PCT Filed: May 18, 2022
(86) PCT No.: PCT/EP2022/063422

§ 371 Date: Nov. 17, 2023

(30) Foreign Application Priority Data
May 19, 2021 (FR) 2105217

(51) Int. Cl. A61K 47/55; A61K 47/54; A61P 35/00; C07D 235/14

(52) U.S. Cl. CPC A61K 47/55; A61K 47/545; A61P 35/00; C07D 235/14

(57) ABSTRACT

The present application relates to novel bifunctional PROTAC-type compounds simultaneously binding the target protein PXR and E3-ubiquitin ligase, to a method for preparing same, and to uses thereof for treating cancers overexpressing PXR.

The present invention relates to the treatment of cancer and more particularly cancers overexpressing the PXR nuclear receptor, such as colorectal cancer.

Colorectal cancer (CRC) is the third most common cancer and is the third cause of death by cancer. Current treatments include surgery, radiation therapy and chemotherapy, sometimes in combination with targeted therapies that demonstrate a minor improvement. However, the efficacy of these treatments is seriously compromised by the frequent appearance of resistance, which leads to the relapse of patients after the treatments stop (50% of patients). In recent years, it has been shown that sub-populations of cancer cells, cancer stem cells (CSCs), were involved in tumor initiation, metastatic development and resistance to drugs, thus leading to tumor recurrence.

The inventors have now demonstrated that the PXR (NR1I2) nuclear receptor is preferentially activated in cancer stem cells and that the extinction of its expression by RNA interference (shRNA) sensitizes this cell population, normally resistant to chemotherapy, and significantly delays tumor recurrence in mice. The inhibition of the PXR (NR112) nuclear receptor therefore makes it possible to sensitize cancer stem cells to current treatments.

The PXR antagonists identified to date (L-sulforaphane, ketoconazole and SAP-70) are, however, either non-specific and/or toxic at the concentrations necessary for the inactivation of PXR, or have not yet been approved for clinical use.

PROTACs (“Proteolysis Targeting Chimeras”) are bifunctional molecules that simultaneously bind a target protein and an E3-ubiquitin ligase. This causes poly-ubiquitination of the target protein which is thus degraded into small peptides and amino acids by the proteasome complex. The PROTAC approach is therefore a chemical protein knock-down strategy

It is therefore desirable to provide bifunctional chimeric ligands capable of inducing targeted proteolysis of PXR according to the PROTAC strategy.

According to first subject matter, the present invention relates to bifunctional compounds conforming to the general formula (I):

L(PXR)-Linker-L(E3 ligase) (I)

wherein:
L(PXR) is a ligand capable of binding to the PXR nuclear receptor,
L(E3 ligase) represents a ligand of the E3-ubiquitin ligase, and
Linker represents a group which makes it possible to covalently bond L(PXR) to L(E3 ligase).

The ubiquitin-proteasome pathway (UPP) is an essential cellular pathway which regulates key regulatory proteins and degrades incorrectly folded or abnormal proteins. The UPP is at the core of several cell processes. If it is defective or imbalanced, it leads to the pathogenesis of various diseases. The covalent attachment of ubiquitin to specific protein substrates is obtained by the action of E3-ubiquitin ligases. These ligases comprise more than 500 different proteins and are classified into several classes defined by the structural element of their E3 functional activity.

The E3 ligase ligand, which constitutes a functional modality of the present compounds, binds to an E3-ubiquitin ligase. The ligase catalyzes the covalent binding of the ubiquitin to the target protein, which in turn induces the degradation of the target protein by the native proteasomes. Thus, the compounds of the present invention are designed in a manner that utilizes the native cell degradation processes but wherein the degradation action is directed to undesirable target proteins that are involved in the etiology of the disease.

Unlike conventional chemical inhibitors, the PROTACs according to the invention act as degradation enzymes with capacity for super-stoichiometric action.

The compounds according to the invention therefore have multiple advantages:
1) they are active at concentrations lower than those of the inhibitor alone, which requires high levels of systemic exposure in order to obtain saturation of the target,
2) they can perform multiple degradation cycles leading to the degradation of the targeted protein,
3) compared to the rapid dissociation kinetics of the inhibitors on their target, the restoration of the protein function after degradation induced by PROTAC requires de novo synthesis of the protein by the cell, which takes a lot longer and thus increases the duration of the effects of the PROTACs.

L(PXR) which is a functional modality of the present compounds that binds to PXR. In some embodiments, the targeting ligand is an analog of PXR JMV6845 ligands:

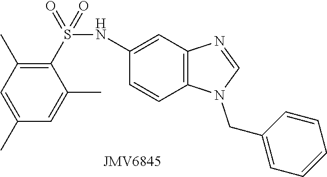 [SMILES: Cc1cc(C)c(S(=O)(=O)Nc2ccc3c(c2)ncn3Cc2ccccc2)c(C)c1]

According to one embodiment, L(PXR) can be selected from the groups of formula (II):

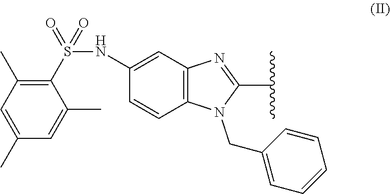 [SMILES: *c1nc2cc(NS(=O)(=O)c3c(C)cc(C)cc3C)ccc2n1Cc1ccccc1]

wherein

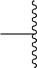

represents the attachment of the group to Linker,

or a pharmaceutically acceptable salt.

Thus, the compounds according to the invention may conform to the following formula (I-1):

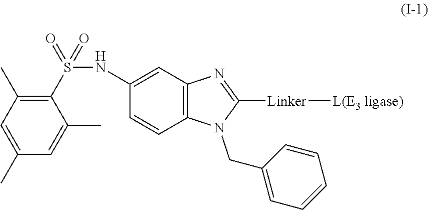

wherein Linker, L(E3 ligase) are as defined hereinbefore or hereinafter, or a pharmaceutically acceptable salt.

According to one embodiment, the E3 ligase ligand binds to cereblon. L(E3 ligase) can especially be selected from:
the groups of formula (IIIA):

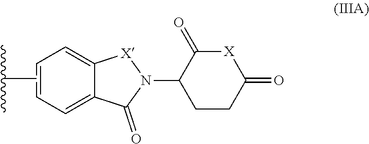

and
the groups of formula (IIIB):

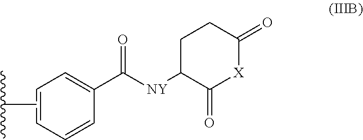

or a pharmaceutically acceptable salt,
in which formulas (IIIA) and (IIIB):
X is NH;
X′ is —C(O)— or —CH₂—;
Y represents H or a C1-C6 alkyl group;

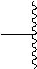

represents the attachment of the group to Linker.

Thus, the compounds according to the invention may especially conform to the formula (I-2) or (I-3):

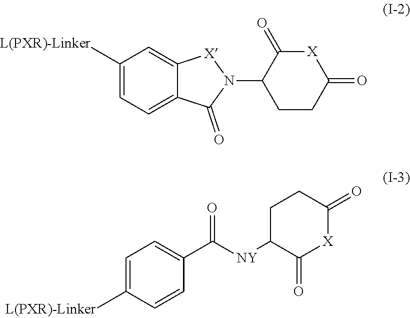

wherein Linker, L(PXR), L(E3 ligase), X, X′, Y are as defined hereinbefore or hereinafter;

or a pharmaceutically acceptable salt.

More particularly, the compounds according to the invention can conform to one of the following formulas (I-4) and (I-5):

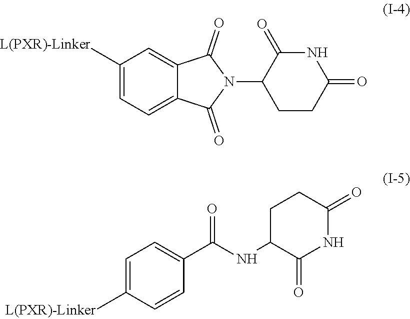 [SMILES: Cc1ccc2c(c1)C(=O)N(C1CCC(=O)NC1=O)C2=O ; Cc1ccc(C(=O)NC2CCC(=O)NC2=O)cc1]

wherein L(PXR), Linker are defined hereinbefore or hereinafter;

or a pharmaceutically acceptable salt.

Linker provides covalent bonding of the targeting ligand with the E3 ligase ligand. According to one embodiment, Linker represents a C1-C20 alkylene group, optionally interrupted or optionally terminating at either and/or both ends, by one of the groups —O—, —S—, —N(R′)—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —S(O)₂—, —OS(O)—, —S(O)O—, —S(O)—, —OS(O)₂—, —N(R′)S(O)₂—, —S(O)₂N(R′)—, —N(R′)S—, —S(O)N(R′)—, —N(R′)S(O)₂N(R′)—, —N(R′)S(O)N(R′)—, C₃-C₁₂ cycloalkylene, 3-to-12-membered heterocyclene comprising 1, 2 or 3 heteroatoms selected from N, O, S, 5-to-12-membered heteroarylene comprising 1, 2 or 3 heteroatoms selected from N, O, S, or any combination thereof, and wherein R′, identical or different, represent H or a C₁-C₆ alkyl group.

Thus, according to a particular embodiment, Linker may be selected from the C4-C20 alkylene groups, optionally interrupted by and/or terminating by one or more groups selected from —NH—, —O—, —C(O)—; piperidinyl, piperazylene.

More particularly, Linker may be represented among the groups of formula (IV):

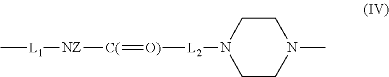

wherein L₁ and L₂, identical or different, represent an alkylene group of 1 to 12 carbon atoms optionally interrupted or terminating by a 3-to-12-membered heterocyclene comprising 1, 2 or 3 heteroatoms selected from N, O, S;

L₁ is bonded to L(PXR) and

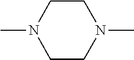

is bonded to L(E3 ligase);

Z represents H or a C₁-C₆ alkyl group.

According to a more particular embodiment, L1 is a C7-alkylene group (—C₇H₁₄—).

According to a more particular embodiment, L₂ is a (C2 to C8)-alkylene group optionally interrupted by a piperidinyl group.

According to one embodiment, the compounds according to the invention may conform to the following formula (V):

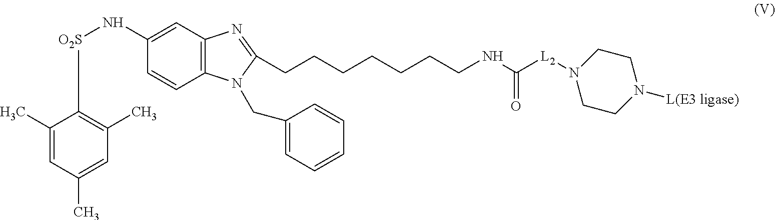

wherein L2 represents a C₂-C₈ linear alkylene group optionally interrupted by a piperidinyl group, and L(E3 ligase) is as defined hereinbefore or hereinafter.

Formulas (I), (II), (IIIA), (IIIB), (IV), (V) represented herein also cover the pharmaceutically acceptable salts thereof, the isotopic derivatives thereof and the stereoisomers thereof.

As used hereinbefore and hereinafter and unless otherwise specified:

“Alkyl” denotes an aliphatic hydrocarbon group which may be linear or branched having about 1 to about 20 carbon atoms in the chain. Preferred alkyl groups have 1 to about 12 carbon atoms in the chain, especially from 1 to 6 carbon atoms. Branched means that one or more lower alkyl groups, such as methyl, ethyl or propyl, are bonded to a linear alkyl chain. “Lower alkyl” means from about 1 to about 4 carbon atoms in the chain which may be linear or branched. The alkyl may be substituted by one or more “alkyl group substituents” which may be identical or different and comprise halo, cycloalkyl, hydroxy, alkoxy, amino, acylamino, aroylamino, carboxy, alkoxycarbonyl, aralkoxycarbonyl, heteroaralkoxycarbonyl or Y¹Y²NCO—, wherein Y¹ and Y² are, independently, hydrogen, optionally substituted alkyl, optionally substituted aryl, optionally substituted aralkyl or optionally substituted heteroaralkyl, or Y¹ and Y², considered together with the N via which Y¹ and Y² are bonded, form a 4 to 7 element heterocyclyl. Typical examples of alkyl groups comprise methyl, trifluoromethyl, cyclopropylmethyl, cyclopentylmethyl, ethyl, n-propyl, i-propyl, n-butyl, t-butyl, n-pentyl, 3-pentyl, methoxyethyl, carboxymethyl, methoxycarbonylethyl, benzyloxycarbonylmethyl, pyridylmethyloxycarbonylmethyl.

“Alkylene” denotes an alkyl group as defined hereinbefore divalent. The preferred alkylene groups are the lower alkylene groups having 1 to about 6 carbon atoms. Typical examples of alkylene groups comprise methylene and ethylene.

“Cycloalkyl” means a non-aromatic, mono- or multicyclic ring system of about 3 to about 10 carbon atoms, preferably of about 5 to about 10 carbon atoms. Preferred ring sizes of the rings of the ring system comprise about 5 to about 6 ring atoms, optionally substituted by one or more substituents. Exemplary monocyclic cycloalkyls comprise cyclopentyl, cyclohexyl, cycloheptyl, and the like. Exemplary multi-cyclic cycloalkyls comprise 1-decalin, norbornyl, adamant-(1 or 2-)yl, and the like.

“Cycloalkylene” means a cycloalkyl group as defined hereinbefore, saturated, divalent, such as especially cyclohexylene.

“Heterocyclyl” means a non-aromatic saturated monocyclic or multicyclic ring system of about 3 to about 10 carbon atoms, preferably of about 5 to about 10 carbon atoms, wherein one or more of the carbon atoms in the ring system is/are one or more heteroelements other than carbon, for example nitrogen, oxygen or sulfur. The preferred ring sizes of the rings of the ring system comprise about 5 to about 6 ring atoms. The designation of aza, oxa or thia as prefix before heterocyclyl defines that at least one nitrogen, oxygen or sulfur atom is present, respectively, as a ring atom. The heterocyclyl may optionally be substituted by one or more substituents, which may be the same or different, and are as defined herein. The nitrogen atom of a heterocyclyl may be a basic nitrogen atom. The nitrogen or sulfur atom of the heterocyclyl may also optionally be oxidized into the corresponding N-oxide, S-oxide or S,S-dioxide. Exemplary monocyclic heterocyclyl rings comprise piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, and the like.

The term “heterocyclene” denotes a heterocyclyl radical as defined hereinbefore divalent.

“Heteroaryl” means a monocyclic or multicyclic aromatic ring system of about 5 to about 14 carbon atoms, preferably of about 5 to about 10 carbon atoms, wherein one or more of the carbon atoms in the ring system is/are one or more heteroelements other than carbon, for example nitrogen, oxygen or sulfur. The preferred ring sizes of the rings of the ring system comprise about 5 to about 6 ring atoms. The “heteroaryl” may also be substituted by one or more substituents. The designation of aza, oxa or thia as a prefix before heteroaryl define that at least one nitrogen, oxygen or sulfur atom is present respectively as a ring atom. A nitrogen atom of a heteroaryl may be a basic nitrogen atom and may also optionally be oxidized to the corresponding N-oxide. Exemplary substituted heteroaryl and heteroaryl groups comprise pyrazinyl, thienyl, isothiazolyl, oxazolyl, pyrazolyl, furazanyl, pyrrolyl, 1,2,4-thiadiazolyl, pyridazinyl, quinoxalinyl, phthalazinyl, imidazo[1,2-a]pyridine, imidazo[2,1-b]thiazolyl, benzofurazanyl, azaindolyl, benzimidazolyl, benzothienyl, thienopyridyl, thienopyrimidinyl, pyrrolopyridyl, imidazopyridyl, benzoazaindol, 1,2,4-triazinyl, benzthiazolyl, furanyl, imidazolyl, indolyl, indolizinyl, isoxazolyl, isoquinolinyl, isothiazolyl, oxadiazolyl, pyrazinyl, pyridazinyl, pyrazolyl, pyridyl, pyrimidinyl, pyrrolyl, quinazolinyl, quinolinyl, 1,3,4-thiadiazolyl, thiazolyl, thienyl, and triazolyl. Preferred heteroaryl groups comprise pyrazinyl, thienyl, pyridyl, pyrimidinyl, isoxazolyl and isothiazolyl.

“Heteroarylene” denotes a heteroaryl radical as defined hereinbefore divalent.

“Substituents” denotes one or more identical or different groups selected from halogen, cyano, cycloalkyl, hydroxy, alkoxy, amino, alkylamino, dialkylamino, aroylamino, carboxy, alkoxycarbonyl, aralkoxycarbonyl, heteroaralkoxycarbonyl.

The compounds of the present invention may be in the form of a free acid or a free base, or a pharmaceutically acceptable salt.

The expression “pharmaceutically acceptable salts” refers to the relatively non-toxic, inorganic and organic acid addition salts, and base addition salts, of the compounds of the present invention. These salts can be prepared in situ during the final isolation and purification of the compounds. In particular, the acid addition salts can be prepared by separately reacting the purified compound in its purified form with an organic or inorganic acid and by isolating the salt thus formed. Among the examples of acid addition salts, are the bromhydrate, chlorhydrate, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptanate, lactobionate, sulfamate, malonate, salicylate, propionate, methylenebis-b-hydroxynaphthoate, gentisic acid, isethionate, di-p-toluoyltartrate, methanesulfonate, ethanesulfonate, benzenesulfonate, p-toluenesulfonate, cyclohexyl sulfamate and quinateslaurylsulfonate salts, and the like. (See for example S. M. Berge et al. “Pharmaceutical Salts” J. Pharm. Sci, 66: pages 1-19 (1977) which is incorporated herein as reference). Acid addition salts can also be prepared by separately reacting the purified compound in its acid form with an organic or inorganic base and by isolating the salt thus formed. Acid addition salts comprise amine and metal salts. Suitable metal salts comprise sodium, potassium, calcium, barium, zinc, magnesium and aluminum salts. Sodium and potassium salts are preferred. Suitable basic inorganic addition salts are prepared from metal bases which comprise sodium hydride, sodium hydroxide, potassium hydroxide, calcium hydroxide, aluminum hydroxide, lithium hydroxide, magnesium hydroxide, zinc hydroxide. Suitable basic addition salts are prepared from amines which have sufficient alkalinity to form a stable salt, and preferably comprise the amines which are often used in medicinal chemistry due to their low toxicity and their acceptability for medical use: ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, for example lysine and arginine, and dicyclohexylamine, and the like.

The compounds of the present invention may have at least one chiral center and may therefore be in the form of a stereoisomer, which, as used herein, encompasses all the isomers of individual compounds which differ only by the orientation of their atoms in space. The term stereoisomer includes the mirror-image isomers (enantiomers that include the (R-) or (S-) configurations of the compounds), the mixtures of mirror-image isomers (physical mixtures of the enantiomers and racemates or racemic mixtures) of geometric compounds (cis/trans or E/Z, R/S isomers) of compounds and isomers of compounds with more than one chiral center which are not mirror images of one another (diastereoisomers). The chiral centers of the compounds can undergo epimerization in vivo; thus, for these compounds, the administration of the compound in its (R-) form is considered to be equivalent to the administration of the compound in its (S-) form. Accordingly, the compounds of the present invention can be manufactured and used in the form of individual isomers and substantially free of other isomers, or in the form of a mixture of various isomers, for example racemic mixtures of stereoisomers.

In some embodiments, the following compounds are suitable for binding to the Cereblon and to PXR:

TABLE 1

| | |
|---|---|
| JMV7158 | 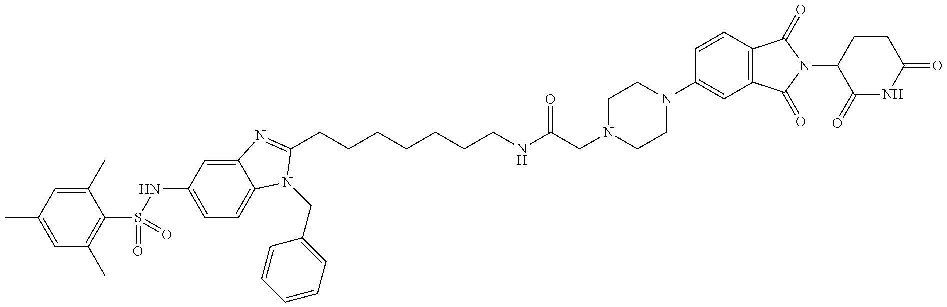 [SMILES: Cc1cc(C)c(S(=O)(=O)Nc2ccc3c(c2)nc(CCCCCCCNC(=O)CN2CCN(c4ccc5c(c4)C(=O)N(C4CCC(=O)NC4=O)C5=O)CC2)n3Cc2ccccc2)c(C)c1] |
| JMV7184 | 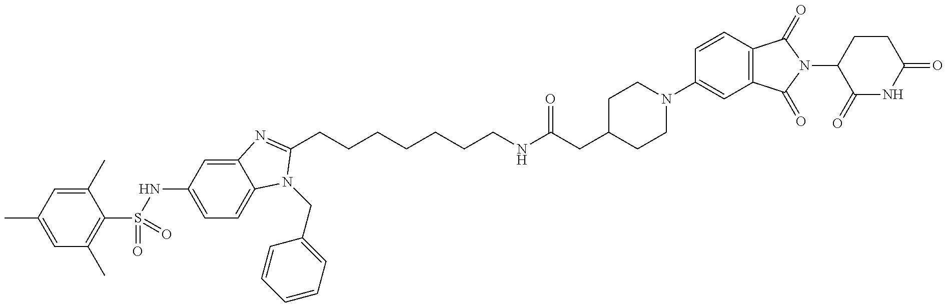 [SMILES: Cc1cc(C)c(S(=O)(=O)Nc2ccc3c(c2)nc(CCCCCCCNC(=O)CC2CCN(c4ccc5c(c4)C(=O)N(C4CCC(=O)NC4=O)C5=O)CC2)n3Cc2ccccc2)c(C)c1] |
| JMV7146 | 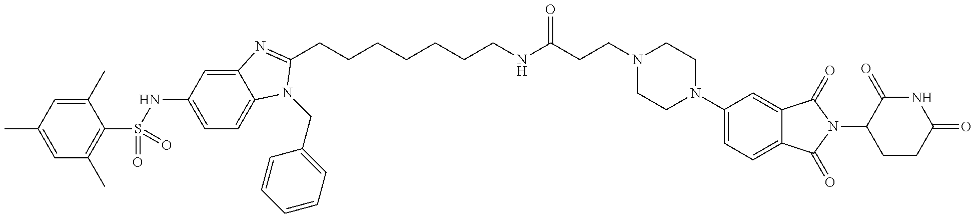 [SMILES: Cc1cc(C)c(S(=O)(=O)Nc2ccc3c(c2)nc(CCCCCCCNC(=O)CCN2CCN(c4ccc5c(c4)C(=O)N(C4CCC(=O)NC4=O)C5=O)CC2)n3Cc2ccccc2)c(C)c1] |
| JMV7154 | 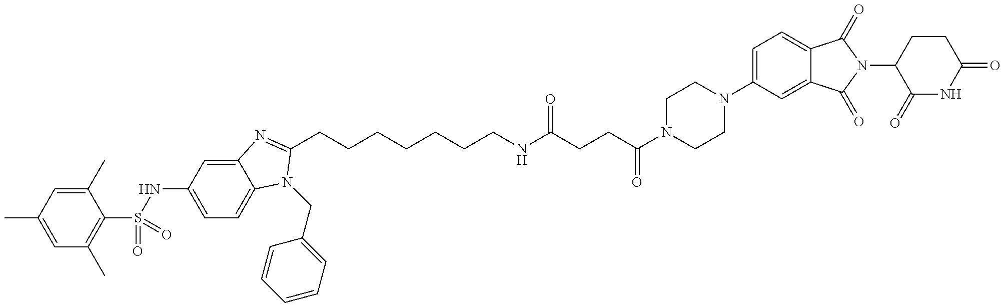 [SMILES: Cc1cc(C)c(S(=O)(=O)Nc2ccc3c(c2)nc(CCCCCCCNC(=O)CCC(=O)N2CCN(c4ccc5c(c4)C(=O)N(C4CCC(=O)NC4=O)C5=O)CC2)n3Cc2ccccc2)c(C)c1] |
| JMV7125 | 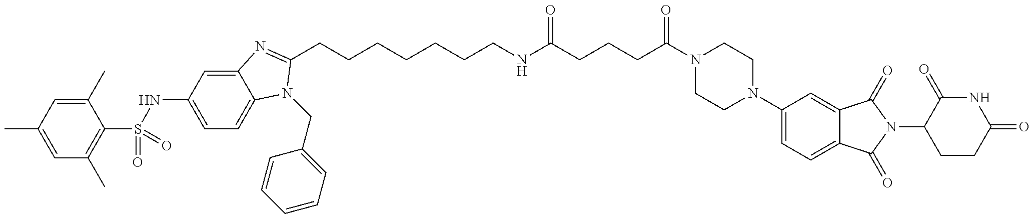 [SMILES: Cc1cc(C)c(S(=O)(=O)Nc2ccc3c(c2)nc(CCCCCCCNC(=O)CCCC(=O)N2CCN(c4ccc5c(c4)C(=O)N(C4CCC(=O)NC4=O)C5=O)CC2)n3Cc2ccccc2)c(C)c1] |
| JMV7048 | 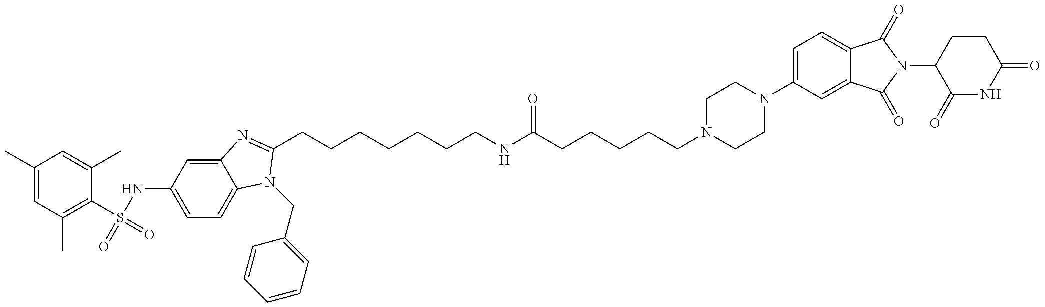 [SMILES: Cc1cc(C)c(S(=O)(=O)Nc2ccc3c(c2)nc(CCCCCCCNC(=O)CCCCCN2CCN(c4ccc5c(c4)C(=O)N(C4CCC(=O)NC4=O)C5=O)CC2)n3Cc2ccccc2)c(C)c1] |
| JMV7505 | 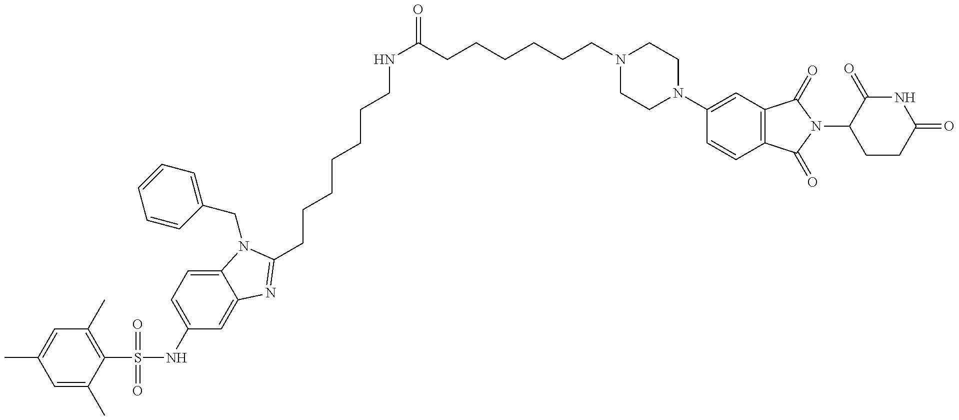 [SMILES: Cc1cc(C)c(S(=O)(=O)Nc2ccc3c(c2)nc(CCCCCCCNC(=O)CCCCCCN2CCN(c4ccc5c(c4)C(=O)N(C4CCC(=O)NC4=O)C5=O)CC2)n3Cc2ccccc2)c(C)c1] |
| JMV7506 | 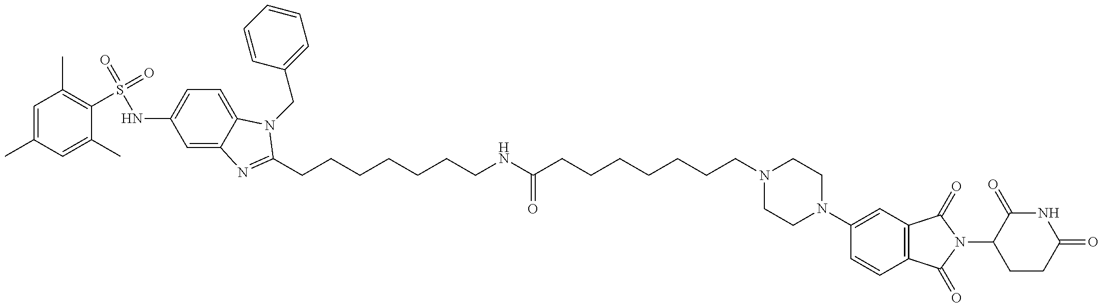 [SMILES: Cc1cc(C)c(S(=O)(=O)Nc2ccc3c(c2)nc(CCCCCCCNC(=O)CCCCCCCN2CCN(c4ccc5c(c4)C(=O)N(C4CCC(=O)NC4=O)C5=O)CC2)n3Cc2ccccc2)c(C)c1] |
| JMV7264 | 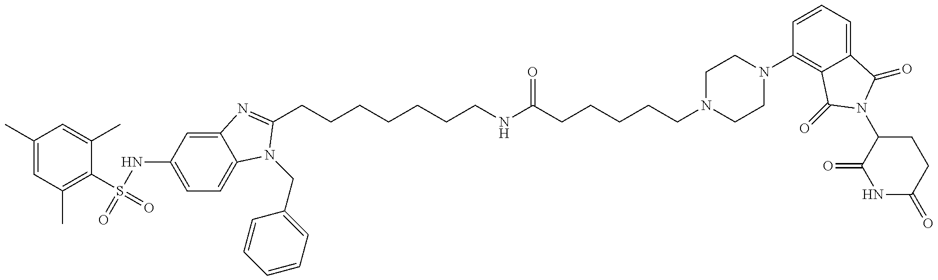 [SMILES: Cc1cc(C)c(S(=O)(=O)Nc2ccc3c(c2)nc(CCCCCCCNC(=O)CCCCCN2CCN(c4cccc5c4C(=O)N(C4CCC(=O)NC4=O)C5=O)CC2)n3Cc2ccccc2)c(C)c1] |
| JMV7605 | 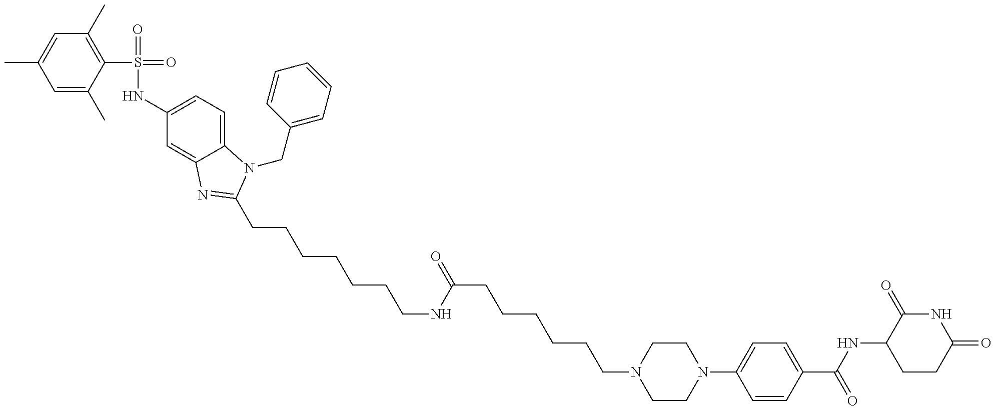 [SMILES: Cc1cc(C)c(S(=O)(=O)Nc2ccc3c(c2)nc(CCCCCCCNC(=O)CCCCCCN2CCN(c4ccc(C(=O)NC5CCC(=O)NC5=O)cc4)CC2)n3Cc2ccccc2)c(C)c1] |
| JMV7727 | 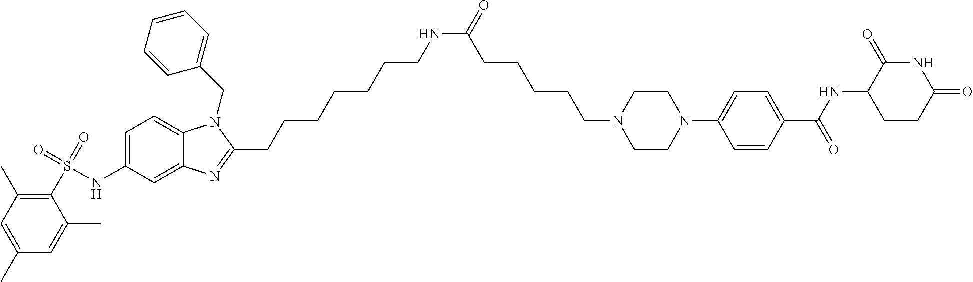 [SMILES: Cc1cc(C)c(S(=O)(=O)Nc2ccc3c(c2)nc(CCCCCCCNC(=O)CCCCCN2CCN(c4ccc(C(=O)NC5CCC(=O)NC5=O)cc4)CC2)n3Cc2ccccc2)c(C)c1] |
| JMV7965 | 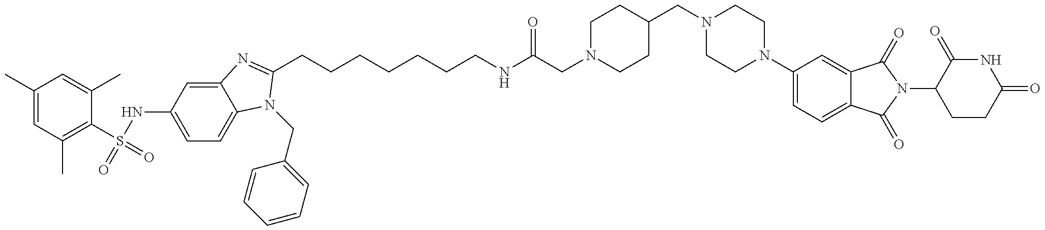 [SMILES: Cc1cc(C)c(S(=O)(=O)Nc2ccc3c(c2)nc(CCCCCCCNC(=O)CN2CCC(CN4CCN(c5ccc6c(c5)C(=O)N(C5CCC(=O)NC5=O)C6=O)CC4)CC2)n3Cc2ccccc2)c(C)c1] |

Most particularly, the compounds according to the invention can be selected from the compounds that conform to one of the following formulas:

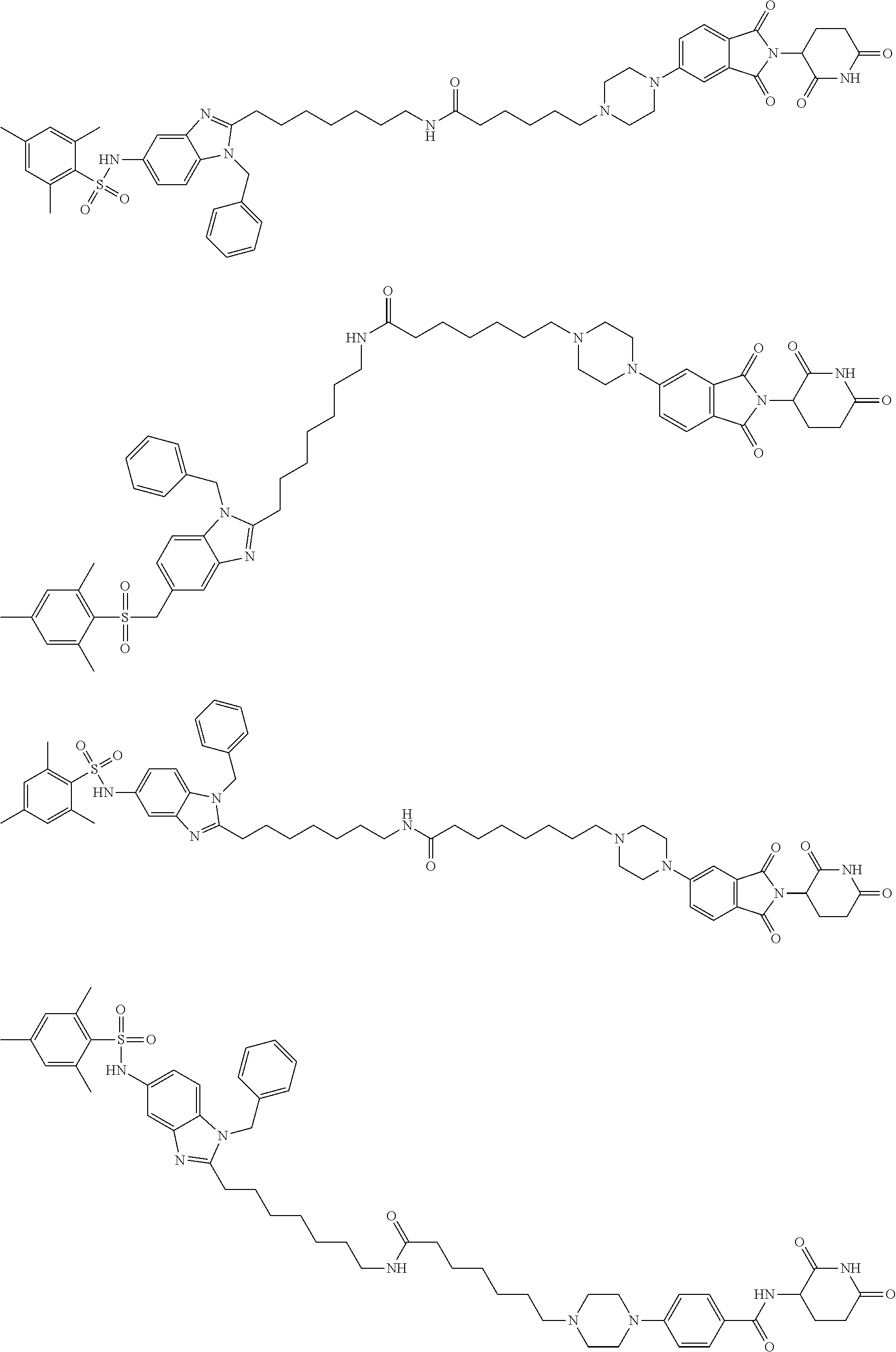 [SMILES: Cc1cc(C)c(S(=O)(=O)Nc2ccc3c(c2)nc(CCCCCCCNC(=O)CCCCCN2CCN(c4ccc5c(c4)C(=O)N(C4CCC(=O)NC4=O)C5=O)CC2)n3Cc2ccccc2)c(C)c1 ; Cc1cc(C)c(S(=O)(=O)Cc2ccc3c(c2)nc(CCCCCCCNC(=O)CCCCCCN2CCN(c4ccc5c(c4)C(=O)N(C4CCC(=O)NC4=O)C5=O)CC2)n3Cc2ccccc2)c(C)c1 ; Cc1cc(C)c(S(=O)(=O)Nc2ccc3c(c2)nc(CCCCCCCNC(=O)CCCCCCCN2CCN(c4ccc5c(c4)C(=O)N(C4CCC(=O)NC4=O)C5=O)CC2)n3Cc2ccccc2)c(C)c1 ; Cc1cc(C)c(S(=O)(=O)Nc2ccc3c(c2)nc(CCCCCCCNC(=O)CCCCCCN2CCN(c4ccc(C(=O)NC5CCC(=O)NC5=O)cc4)CC2)n3Cc2ccccc2)c(C)c1]

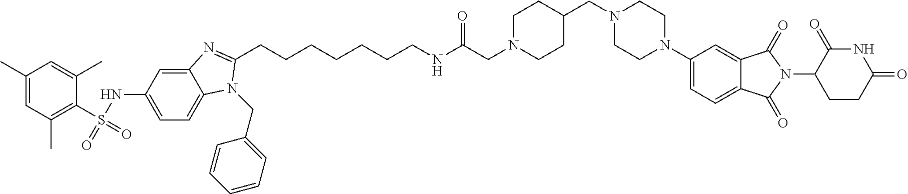 [SMILES: Cc1cc(C)c(S(=O)(=O)Nc2ccc3c(c2)nc(CCCCCCCNC(=O)CN2CCC(CN4CCN(c5ccc6c(c5)C(=O)N(C5CCC(=O)NC5=O)C6=O)CC4)CC2)n3Cc2ccccc2)c(C)c1]

According to further subject matter, the present invention also relates to the method for preparing a compound according to the invention.

The compounds of general formula (I) can be prepared by application or adaptation of any method known per se and/or within the reach of a skilled person, especially those described by Larock in Comprehensive Organic Transformations, VCH Pub., 1989, or by applying or adapting the methods described in the following examples.

According to the invention, said method comprises the coupling of a compound of formula (B) and of a compound of formula (C):

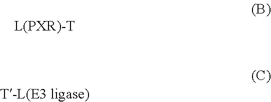

such that L(PXR) and L(E3 ligase) are as defined hereinbefore, and T and T′ are two groups of Linker precursors, that is to say of which the coupling makes it possible to lead to the Linker group, such that they each respectively have a complementary reactive terminal function.

Herein “complementary reactive functions” denotes two functions capable of reacting together to form a function ensuring a covalent bond between T and T′. Thus, typically, T and T′ are such that T has an amine-type terminal function and T′ has a terminal function of the carboxylic acid type.

Thus, typically, T represents a group of formula (T-B):

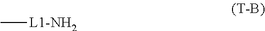

and T′ represents a group of formula (T-C):

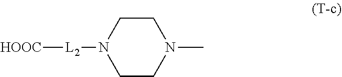

wherein L₁ and L₂ are as defined hereinbefore.

Said coupling can advantageously be carried out in the presence of a peptide coupling agent such as BOP (benzotriazol-1-yloxytris(dimethylamino)phosphonium hexafluorophosphate), typically in the presence of an organic base such as Hünig's base N,N-diisopropylethylamine (DIPEA or DIEA).

According to one embodiment, the compound (B) conforms to the formula (A):

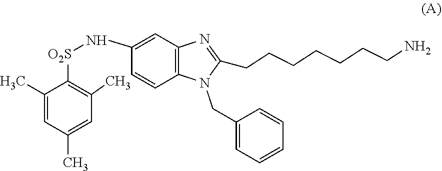 [SMILES: Cc1cc(C)c(S(=O)(=O)Nc2ccc3c(c2)nc(CCCCCCCN)n3Cc2ccccc2)c(C)c1]

According to one embodiment, the compound (C) conforms to the formula (C-1):

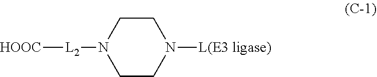

wherein L₂ and L(E3 ligase) are as defined hereinbefore.

Optionally, said method may also comprise the step consisting of isolating the product of formula (I) obtained.

In the reactions described hereinafter, it may be necessary to protect the reactive functional groups, for example the hydroxy, amino, imino, thio, carboxy groups, when they are desired in the final product, to avoid their undesirable participation in the reactions. Conventional protection groups can be used in accordance with standard practice, for example, see T. W. Green and P. G. M. Wuts in Protective Groups in Organic Chemistry, John Wiley and Sons, 1991; J. F. W. McOmie in Protective Groups in Organic Chemistry, Plenum Press, 1973.

The compound thus prepared can be recovered from the mixture of the reaction by conventional means. For example, the compounds can be recovered by distilling the solvent of the mixture of the reaction or if necessary after distillation of the solvent of the mixture of the solution, by pouring the remainder into water followed by extraction with an organic solvent immiscible in water, and by distilling the solvent from the extract. In addition, if desired, the product may be further purified by various techniques, such as recrystallization, reprecipitation or various chromatography techniques, especially column chromatography or preparative thin-film chromatography.

It can be seen that the useful compounds according to the present invention may contain asymmetric centers. These asymmetric centers can be independently in R or S configuration. It will be obvious to a skilled person that certain useful compounds according to the invention can also have geometric isomerism. It should be understood that the present invention comprises individual geometric isomers and stereoisomers and mixtures thereof, including racemic mixtures, of compounds of formula (I) hereinbefore. Isomers of this type may be separated from their mixtures, by applying or adapting known methods, for example chromatography techniques or recrystallization techniques, or they are prepared separately from the appropriate isomers of their intermediates.

The base products or reagents used are commercially available and/or can be prepared by the application or adaptation of known methods, for example methods as described in the Reference Examples or their obvious chemical equivalents.

The method according to the invention can implement the intermediate of formula (A) which is novel.

According to further subject matter, the present invention therefore also relates to the compound of formula (A):

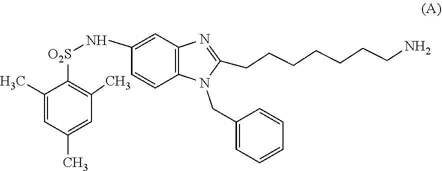 [SMILES: Cc1cc(C)c(S(=O)(=O)Nc2ccc3c(c2)nc(CCCCCCCN)n3Cc2ccccc2)c(C)c1]

The compound of formula (A) can be prepared by coupling the following compounds:

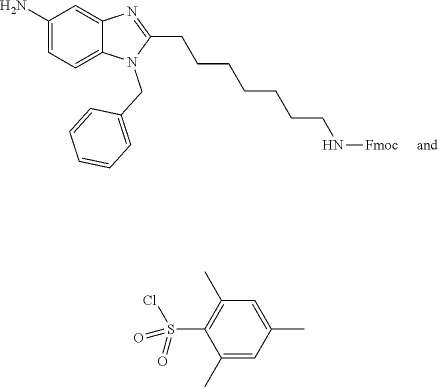

This coupling can typically be carried out by application or adaptation of the procedure described in example 1.

According to the present invention, the compounds of formula (I) are capable of inducing the targeted proteolysis of PXR. The compounds of formula (I) are therefore useful in the treatment and/or prevention of cancers, especially cancers overexpressing PXR.

The present invention therefore also relates to pharmaceutical compositions comprising a compound according to the invention with a pharmaceutically acceptable excipient.

Preferably, said composition contains an effective amount of the compound according to the invention.

According to further subject matter, the present invention also relates to a compound of general formula (I) for the treatment and/or prevention of cancers, especially cancers overexpressing PXR.

Cancers overexpressing PXR are especially colorectal cancer, and pancreatic, liver and breast cancers.

Typically, the compounds according to the invention can be used in combination with an anti-cancer agent. Such anti-cancer agents can especially be selected from 5-Fluorouracil (5-FU), Irinotecan (CPT11), Oxaliplatin, Cisplatin, Tamoxifen, Paclitaxel, Doxorubicin, Vonblastin, Cyclophosphamide (CPA), Isophosphamide (IFO).

Preferably, said composition is administered to a patient in need thereof. Said patient is especially a patient resistant to the above-mentioned anti-cancer agents.

The type of formulation of the pharmaceutical compositions of the invention depends on the mode of administration, which may include an injection that can be enteral (for example, oral), parenteral (for example, subcutaneous (sc), intravenous (iv), intramuscular (im) and intrasternal), or infusion techniques, which can be intravenous or arterial, intramedullary, intrathecal, intraventricular, transdermal, interdermal, rectal, intravaginal, intraperitoneal, topical mucous membrane, nasal, oral, sublingual, intratracheal instillation, bronchial instillation and/or inhalation. Generally, the most suitable administration pathway depends on various factors, especially the nature of the agent (for example, its stability in the environment of the digestive tract) and/or the state of the subject (for example, if the subject is capable of tolerating oral administration). In some embodiments, the compositions are formulated for oral or intravenous administration (for example, systemic intravenous injection).

The expression “pharmaceutically acceptable carrier”, as known in the art, denotes a pharmaceutically acceptable material, composition or carrier, suitable for the administration of compounds of the present invention to mammals. Suitable supports may include, for example, liquids (both aqueous and non-aqueous and combinations thereof), solids, encapsulating materials, gases and combinations thereof (for example, semi-solids), which operate to transport or transport the compound from one organ or body part to another organ or body part. A support is “acceptable” in the sense that it is physiologically inert and compatible with the other components of the formulation and which is non-toxic for the subject or patient. Based on the type of formulation,

As a result, the compounds of formula I may be formulated as solid compositions (for example, powders, tablets, dispersible granules, capsules, wafers and suppositories), liquid compositions (for example, solutions in which the compound is dissolved, suspensions in which the particles of the compound are dispersed, emulsions and solutions containing liposomes, micelles or nanoparticles, syrups and elixirs); semi-solid compositions (for example, gels, suspensions and creams); and gases (for example, propellants for aerosol compositions). The compounds may also be formulated for rapid, intermediate or prolonged release.

The excipients which are suitable for solid administrations are derivatives of cellulose or microcrystalline cellulose, alkaline earth carbonates, magnesium phosphate, starches, modified starches, lactose for solid forms. For parenteral use, water, aqueous solutes, physiological serum, isotonic solutes are the carriers most conveniently used.

The dosage can vary within the large limits based upon the therapeutic indication and the administration pathway, as well as the age and weight of the subject.

### FIGURES

FIG. 1 depicts the PXR affinity of pre-PROTAC JMV6944 measured by RT-FRET.

FIG. 2 illustrates the activation of PXR by the pre-PROTAC JMV6944 and the PROTACs that result therefrom as measured by a luciferase reporter gene placed under the control of the CYP3A4 promoter, the target gene of PXR.

FIGS. 3A and 3B depict the induction of a target gene of PXR (i.e. CYP3A4) by the pre-PROTAC JMV6944 and the PROTACs resulting therefrom measured by RT-qPCR.

FIGS. 4A and 4B illustrate and depict the effect of the PROTACs JMV7048 and JMV7965 on the induction of CYP34 by western blotting.

FIGS. 5A and 5B illustrate the effects of the PROTACs on cell viability in various cell lines (LS174T, FIT29) and primo-culture (CRC1) derived from colon cancer.

FIGS. 6A-E depict the effects of the PROTACs on the degradation of the PXR protein in the LS174T cells, measured by western blotting.

FIGS. 7A and 7B respectively depict the effects of the PROTACs on the degradation of the PXR protein in the FIEPG2 (7A) and ASPC1 (7B) cells, measured by western blotting.

FIGS. 8A-B illustrate the importance of the proteasome pathway in the effects of the PROTACs on the degradation of the PXR protein measured by western blotting.

FIGS. 9A-C respectively depict the effect of JMV7048 on the degradation of the PXR protein in vivo, on xenografts of LS174T cells in SCID mice.

FIGS. 10A-D respectively depict the effects of the PROTACs on the population of cancer stem cells: inhibition of ALDFI activity (10A), inhibition of their self-renewal capacity (10B) and sensitization to chemotherapy (10C and 10D)

FIG. 11 illustrates the interaction mode of JMV6944 with the LBD of hPXR. (11A) Entire structure of the complex. The activating helix H12 is indicated. The arrow symbolizes the extension of the PROTACs synthesized subsequently. (11B) Enlargement of the output pathway of JMV6944 and superimposition with the structure of the hPXR-LBD/SR12813 complex. The end of the H2′ helix, residues 206 to 209, rearranges in the presence of the ligand. (11C) Interactions of JMV6944 with the residues of the hPXR binding pocket residues and depiction of the electron density of the ligand (omit type difference map).

The following examples illustrate the invention, without however limiting it. The starting products used are products known or prepared according to known procedures.

The compounds of the present invention will be better understood in connection with the synthesis diagrams described in various examples of work and which illustrate non-limiting processes by which the compounds of the invention can be prepared. The percentages are expressed by weight, unless otherwise indicated.

### EXAMPLE 1: SYNTHESIS OF JMV6944

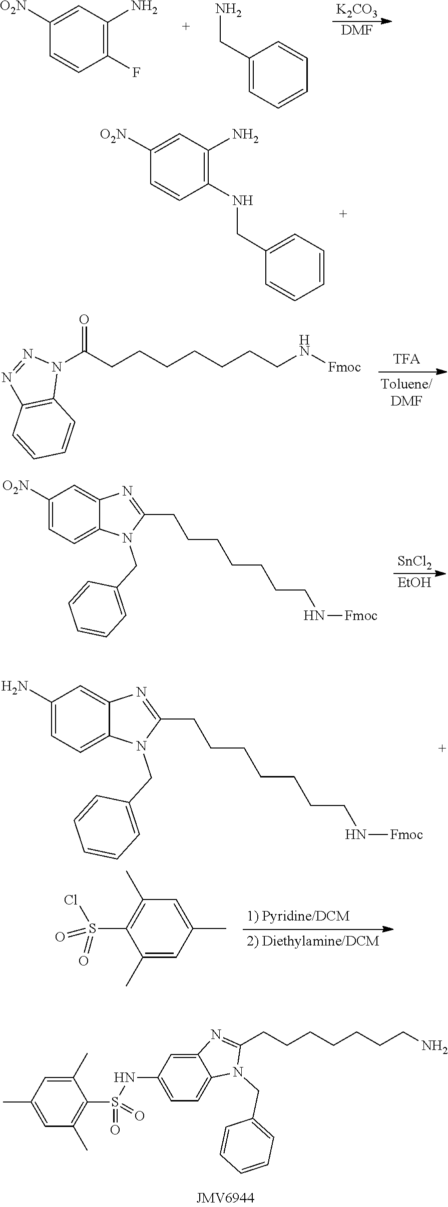

### Step 1: N1-benzyl-4-nitrobenzene-1,2-diamine

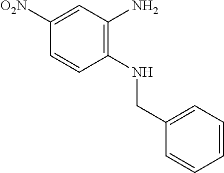 [SMILES: Nc1cc([N+](=O)[O-])ccc1NCc1ccccc1]

K2CO3 (13.28 g, 96.08 mmol) is added to a solution containing 2-fluoro-5-nitroaniline (5 g, 32.03 mmol) and benzylamine (7.01 ml, 64.05 mmol) in DMF (50 ml). The reaction medium is stirred for 24 h at 100° C. The reaction medium is diluted in an ethyl acetate/H2O mixture. The organic phase is washed successively with water, 1N KHSO4, saturated NaCl and dried over magnesium sulphate. After evaporation, the product is triturated in diethyl ether and drained. The compound 1 N1-benzyl-4-nitrobenzene-1,2-diamine is obtained in the form of a yellow solid with a mass of 7.5 g (96% yield). ESI: M+H 244.1.

### Step 2: (9H-fluoren-9-yl)methyl N-{2-[4-(1-benzyl-5-nitro-1H-1,3-benzodiazol-2-yl)butoxy]ethyl}carbamate

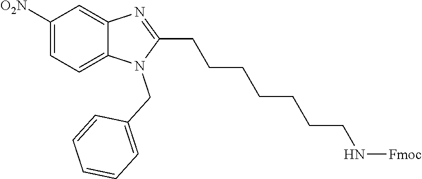

TFA (0.91 ml, 12.28 mmol) is added to a solution containing N1-benzyl-4-nitrobenzene-1,2-diamine (0.747 g, 3.07 mmol) and (9H-fluoren-9-yl)methyl N-[8-(1H-1,2,3-benzotriazol-1-yl)-8-oxooctyl]carbamate (1.63 g, 3.37 mmol) in toluene/DMF (9/1) mixture (45 ml/5 ml). The reaction medium is stirred for 6 h at 60° C. The reaction medium is cooled to room temperature and then to 0° C. The solid is drained and then washed twice with diethyl ether. The powder is dissolved in acetic acid and heated to 100° for 18 h. After evaporation, the compound 2 (9H-fluoren-9-yl)methyl N-{2-[4-(1-benzyl-5-nitro-1H-1,3-benzodiazol-2-yl)butoxy]ethyl}carbamate is obtained in the form of a yellow oil 0.55 g (30% yield). ESI: M+H 589.2.

### Step 3: (9H-fluoren-9-yl)methyl N-[7-(5-amino-1-benzyl-1H-1,3-benzodiazol-2-yl)heptyl]carbamate

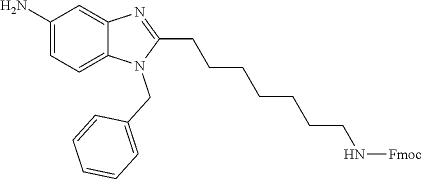

SnCl2 (1.2 g, 6.34 mmol) is added to a solution containing (9H-fluoren-9-yl)methyl N-{2-[4-(1-benzyl-5-nitro-1H-1,3-benzodiazol-2-yl)butoxy]ethyl}carbamate (0.75 g, 1.27 mmol) in ethanol (30 ml). The reaction medium is stirred for 2 h at 80° C. The reaction medium is diluted in a mixture of ethyl acetate/NaHCO₃ and filtered through celite. The organic phase is recovered and dried on MgS04. After evaporation, the compound 3 (9H-fluoren-9-yl)methyl N-[7-(5-amino-1-benzyl-1H-1,3-benzodiazol-2-yl)heptyl]carbamate is obtained in the form of a yellow powder 0.55 g (yield 77%). ESI: M+H 559.3.

### Step 4: N-[2-(7-aminoheptyl)-1-benzyl-1H-1,3-benzodiazol-5-yl]-2,4,6-trimethylbenzene-1-sulfonamide

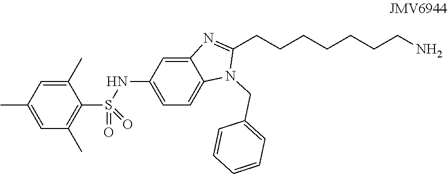 [SMILES: Cc1cc(C)c(S(=O)(=O)Nc2ccc3c(c2)nc(CCCCCCCN)n3Cc2ccccc2)c(C)c1]

2-mesitylenesulfonyl chloride (0.166 g, 0.75 mmol) per portion is added to a solution containing (9H-fluoren-9-yl)methyl N-[7-(5-amino-1-benzyl-1H-1,3-benzodiazol-2-yl) heptyl]carbamate (0.386 g, 0.69 mmol) in a pyridine/DCM (1/1) mixture (5 ml/5 ml) at 0° C. The reaction medium is brought to room temperature and stirred for 18 h. Diethylamine (2 ml) is added to the reaction medium and stirred for 2 h. The solution is concentrated under reduced pressure. The oil obtained is purified by preparative HPLC. After lyophilization, a yellow powder is obtained 0.201 g (56% yield). ESI: M+H 519.4. ¹H NMR (600 MHz, DMSO-d6): δ 10.53 (s, 1H), 7.77 (m, 3H), 7.64 (d, J=8.92 Hz, 1H), 7.33 (m, 4H), 7.20 (d, J=6.81 Hz, 2H), 7.10 (dd, J=1.79, 8.88 Hz, 1H), 7.01 (s, 2H), 5.63 (s, 2H), 3.08 (m, 2H), 2.75 (m, 2H), 2.58 (s, 6H), 2.21 (s, 3H), 1.66 (m, 2H), 1.48 (m, 2H), 1.26 (m, 6H).

13C NMR (125 MHz, DMSO-d6): δ 155.3, 142.7, 139.2, 135.8, 135.4, 133.9, 132.3, 129.4, 129.3, 129.3, 128.5, 127.3, 117.7, 113.7, 47.7, 39.4, 39.2, 28.6, 28.4, 27.3, 26.5.

### EXAMPLE 2: SYNTHESIS OF JMV7048

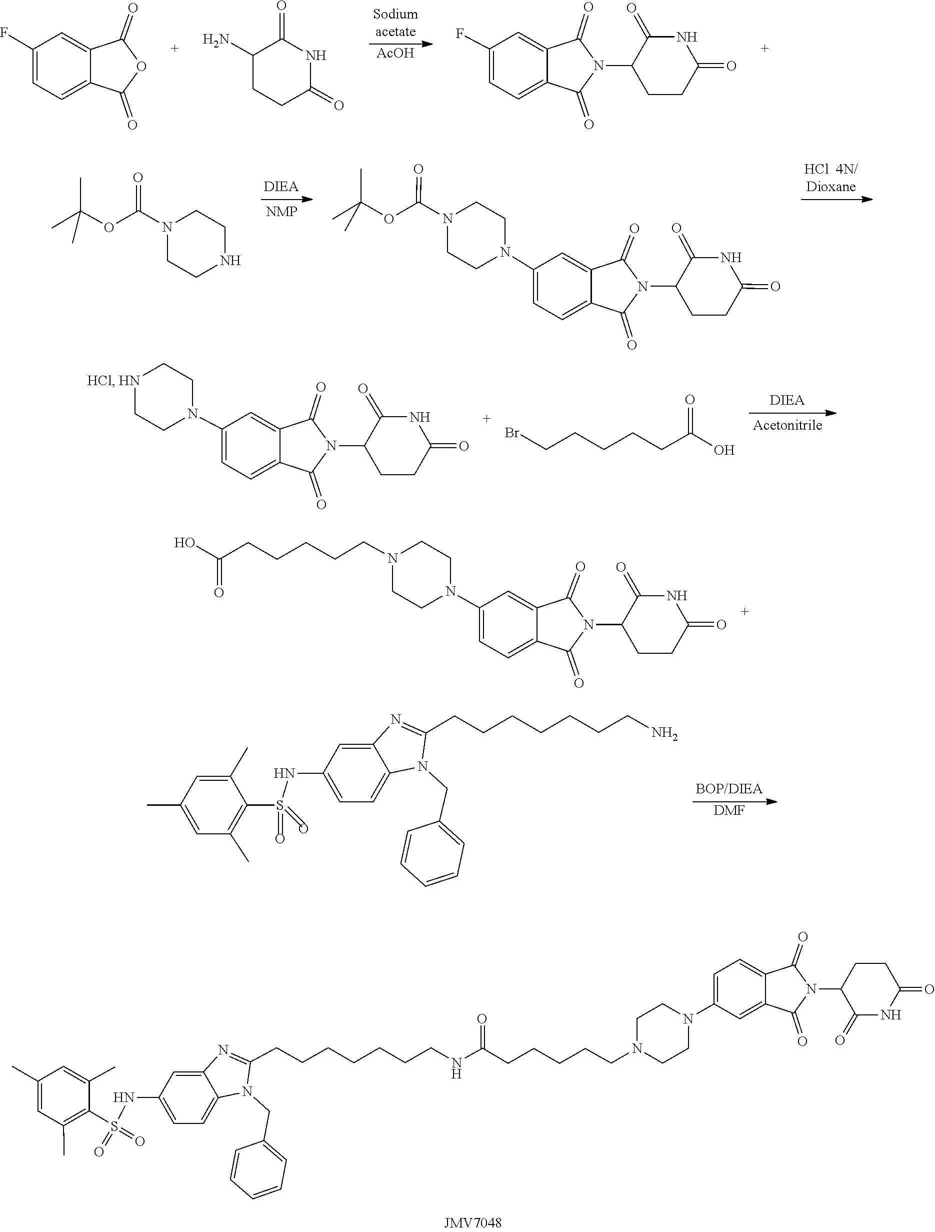

### Step 1: 2-(2,6-dioxopiperidin-3-yl)-5-fluoroisoindole-1,3-dione

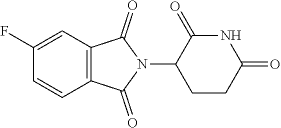 [SMILES: O=C1CCC(N2C(=O)c3ccc(F)cc3C2=O)C(=O)N1]

The reaction medium containing 4-fluorophthalic anhydride (2.43 g, 14.63 mmol) and 3-aminopiperidine-2,6-dione (2.38 g, 14.63 mmol) and sodium acetate (2.4 g, 29.26 mmol) in acetic acid (50 ml) is heated to 100° C. for 24 h. After cooling to room temperature, add water (150 mL) to the reaction mixture, drain the mixture and wash with ether several times. Place in the desiccator overnight at 50° C., the compound 1,2-(2,6-dioxopiperidin-3-yl)-5-fluoroisoindoline-1,3-dione is obtained in the form of a pink solid with a mass of 4 g (yield 99%) ESI: M+H 277.2. ¹H NMR (600 MHz, DMSO-d₆): δ 11.15 (s, 1H), 8.03-8.00 (dd, J=4.59, 8.02 Hz, 1H), 7.87-7.85 (dd, J=2.29, 8.02 Hz, 1H), 7.75-7.71 (t, J=2.29, 4.59, 8.02 Hz, 1H), 5.19-5.16 (dd, J=5.51, 13.03, 1H), 2.94-2.87 (m, 1H), 2.64-2.59 (m, 1H), 2.58-2.51 (m, 1H), 2.10-2.05 (m, 1H); ¹³C NMR (125 MHz, DMSO-d₆) δ 173.2, 173.2, 170.2, 170.1, 167.4, 166.6, 166.6, 166.3, 165.4, 134.7, 134.6, 127.9, 126.7, 126.7, 122.3, 122.1, 112.0, 111.8, 49.6, 31.3, 22.4.

### Step 2: tert-butyl 4-(2-(2,6-dioxopiperidin-3-yl)-1,3-dioxoindolin-5-yl)piperazine-1-carboxylate

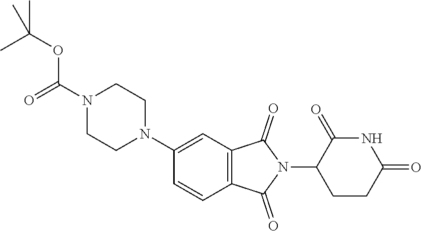 [SMILES: CC(C)(C)OC(=O)N1CCN(c2ccc3c(c2)C(=O)N(C2CCC(=O)NC2=O)C3=O)CC1]

The compound 2-(2,6-dioxopiperidin-3-yl)-5-fluoroisoindoline-1,3-dione (500 mg, 1.81 mmol) is dissolved in NMP (7 ml) at room temperature. DIEA (0.89 ml, 5.43 mmol) and t-butyl 1-piperazine-carboxylate (370.9 mg, 1.99 mmol) are added and the mixture is stirred at 140° C. for 24 h. The solution is diluted in water (100 ml). Extract twice with ethyl acetate and the organic phase is washed with saturated NaCl and dried over magnesium sulfate. After evaporation, the oil obtained is purified on silica gel with a petroleum ether/ethyl acetate eluent (3/1). Tert-butyl 4-(2-(2,6-dioxopiperidin-3-yl)-1,3-dioxoindolin-5-yl)piperazine-1-carboxylate is obtained in the form of a yellow solid with a mass of 655 mg (yield 82%). ESI: M+H 443.1. ¹H NMR (600 MHz, DMSO-d6): δ 11.09 (s, 1H), 7.70 (d, J=8.56 Hz, 1H), 7.35 (d, J=2.08 Hz, 1H), 7.26-7.24 (dd, J=2.08, 8.56 Hz, 1H), 5.08 (m, 1H), 3.47 (s, 8H), 2.93-2.86 (m, 1H), 2.61-2.48 (m, 2H), 2.03 (m, 1H), 1.43 (s, 9H). 13C NMR (125 MHz, DMSO-d6) δ 173.2, 170.5, 167.9, 167.4, 155.4, 154.3, 134.3, 125.3, 119.0, 118.3, 108.5, 79.6, 49.2, 47.0, 31.4, 28.5, 22.6.

### Step 3: 2-(2,6-dioxopiperidin-3-yl)-5-(piperazin-1-yl)isoindoline-1,3-dione

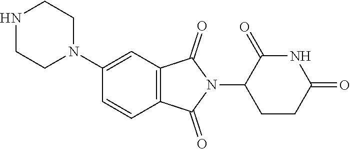 [SMILES: O=C1CCC(N2C(=O)c3ccc(N4CCNCC4)cc3C2=O)C(=O)N1]

The tert-butyl 4-(2-(2,6-dioxopiperidin-3-yl)-1,3-dioxoindolin-5-yl)piperazine-1-carboxylate (464 mg, 1.04 mmol) compound is dissolved in a 4N solution of HCl in dioxane (4 ml) and the reaction medium is stirred at room temperature for 2 hours and then concentrated and triturated with ether. The solid obtained in the form of a yellow powder with a mass of 323 mg (90% yield). ESI: M+H 343.1. ¹H NMR (600 MHz, DMSO-d6): δ 11.09 (s, 1H), 9.71 (m, 2H), 7.73 (d, J=8.61 Hz, 1H), 7.44 (d, J=2.08 Hz, 1H), 7.32 (dd, J=2.08, 8.61 Hz, 1H), 5.09 (m, 1H), 3.73 (m, 4H), 3.19 (m, 4H), 2.89 (m, 1H), 2.61-2.48 (m, 2H), 2.03 (m, 1H). 13C NMR (125 MHz, DMSO-d6) δ 173.2, 170.4, 167.8, 167.3, 154.8, 134.2, 125.4, 120.0, 119.0, 109.2, 49.2, 44.5, 42.4, 31.4, 22.6.

### Step 4: 6-(4-(2-(2,6-dioxopiperid-3-yl)-1,3-dioxoindolin-5-yl)piperazin-1-yl) hexanoic Acid

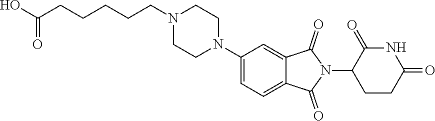 [SMILES: O=C(O)CCCCCN1CCN(c2ccc3c(c2)C(=O)N(C2CCC(=O)NC2=O)C3=O)CC1]

The compound 2-(2,6-dioxopiperidin-3-yl)-5-(piperazin-1-yl)isoindoline-1,3-dione (100 g, 0.29 mmol) is dissolved in acetonitrile (5 ml). 6-bromohexanoic acid (152 mg, 0.73 mmol) and DIEA (0.193 ml, 1.16 mmol) are added and the mixture is stirred at 60° C. for 24 h. The solution is concentrated under reduced pressure. The oil obtained is purified by preparative HPLC. After lyophilization, a yellow powder is obtained with a mass of 90 mg (65% yield). ESI: M+H 457.3. ¹H NMR (600 MHz, DMSO-d6): δ 12.08 (m, 1H), 11.04 (s, 1H), 9.73 (m, 1H), 7.77 (d, J=8.50 Hz), 7.50 (d, J=1.90 Hz), 7.37 (dd, J=1.90, 8.50 Hz), 5.10 (m, 1H), 4.23 (m, 2H), 3.59 (m, 2H), 3.25 (m, 2H), 3.14 (m, 4H), 2.90 (m, 1H), 2.59 (m, 2H), 2.25 (m, 2H), 2.04 (m, 1H), 1.69 (m, 2H), 1.55 (m, 2H), 1.33 (m, 2H). 13C NMR (125 MHz, DMSO-d6) δ 174.7, 173.2, 170.4, 167.8, 167.3, 154.6, 134.2, 125.4, 120.4, 119.2, 109.4, 55.7, 50.7, 49.3, 44.8, 33.7, 31.4, 25.9, 24.3, 23.4.

### Step 5: JMV7048

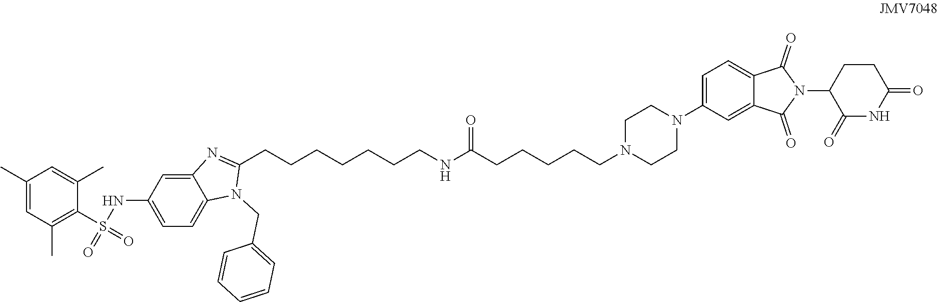 [SMILES: Cc1cc(C)c(S(=O)(=O)Nc2ccc3c(c2)nc(CCCCCCCNC(=O)CCCCCN2CCN(c4ccc5c(c4)C(=O)N(C4CCC(=O)NC4=O)C5=O)CC2)n3Cc2ccccc2)c(C)c1]

BOP (52 mg, 0.12 mmol) is added to a solution containing N-[2-(7-aminoheptyl)-1-benzyl-1H-1,3-benzodiazol-5-yl]-2,4,6-trimethylbenzene-1-sulfonamide (41 mg, 0.079 mmol) (example 1, JMV6944), 6-(4-(2-(2,6-dioxopiperidin-3-yl)-1,3-dioxoisoindol-5-yl)piperazin-1-yl)hexanoic acid (34 mg, 0.079 mmol) and DIEA (0.039 ml, 0.237 mmol) in DMF (5 ml). The reaction medium is stirred for two hours at room temperature. The solution is concentrated under reduced pressure. The oil obtained is purified by preparative HPLC. After lyophilization, a yellow powder is obtained with a mass of 52 mg (66% yield). ESI: M+H 958.0. ¹H NMR (600 MHz, DMSO-d6): δ 11.02 (s, 1H), 10.42 (m, 1H), 9.78 (m, 1H), 7.69 (d, J=8.49 Hz, 1H), 7.65 (m, 1H), 7.54 (d, J=8.89 Hz, 1H), 7.41 (d, J=2.01 Hz, 1H), 7.29-7.20 (m, 5H), 7.11 (m, 2H), 7.00 (dd, J=2.01, 8.89 Hz, 1H), 6.93 (s, 2H), 5.02 (dd, J=5.53, 13.14 Hz, 1H)

### EXAMPLE 3: SYNTHESIS OF JMV7505

### Step 1: 7-{4-[2-(2,6-dioxopiperidin-3-yl)-1,3-dioxo-2,3-dihydro-1H-isoindol-5-yl] piperazin-1-yl}heptanoic Acid

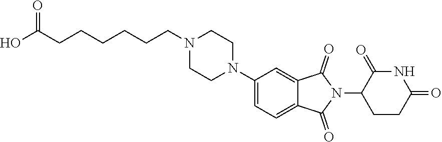 [SMILES: O=C(O)CCCCCCN1CCN(c2ccc3c(c2)C(=O)N(C2CCC(=O)NC2=O)C3=O)CC1]

Compound 2-(2,6-dioxopiperidin-3-yl)-5-(piperazin-1-yl)isoindoline-1,3-dione (100 mg, 0.29 mmol) (example 2, step 3) is dissolved in acetonitrile (5 ml). 7-bromoheptanoic acid (155 mg, 0.73 mmol) and DIEA (0.193 ml, 1.16 mmol) are added and the mixture is stirred at 60° C. for 24 h. The solution is concentrated under reduced pressure. The oil obtained is purified by preparative HPLC. After lyophilization, a yellow powder is obtained with a mass of 93 mg (65% yield). ESI: M+H 471.3.

### Step 2

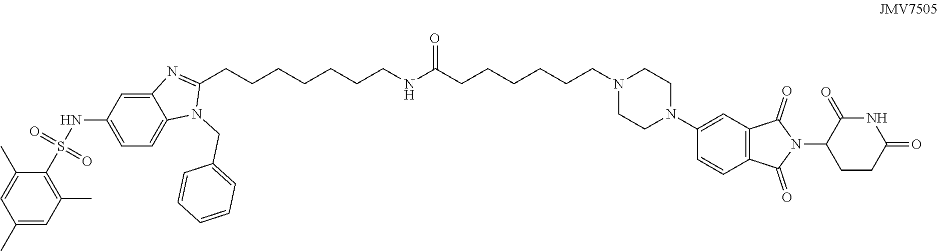 [SMILES: Cc1cc(C)c(S(=O)(=O)Nc2ccc3c(c2)nc(CCCCCCCNC(=O)CCCCCCN2CCN(c4ccc5c(c4)C(=O)N(C4CCC(=O)NC4=O)C5=O)CC2)n3Cc2ccccc2)c(C)c1]

BOP (52 mg, 0.12 mmol) is added to a solution containing N-[2-(7-aminoheptyl)-1-benzyl-1H-1,3-benzodiazol-5-yl]-2,4,6-trimethylbenzene-1-sulfonamide (41 mg, 0.079 mmol) (example 1, JMV6944), 6-(4-(2-(2,6-dioxopiperidin-3-yl)-1,3-dioxoindolin-5-yl)piperazin-1-yl)heptanoic acid (34 mg, 0.079 mmol) and DIEA (0.039 ml, 0.237 mmol) in DMF (5 ml). The reaction medium is stirred for two hours at room temperature. The solution is concentrated under reduced pressure. The oil obtained is purified by preparative HPLC. After lyophilization, a white powder is obtained with a mass of 52 mg (yield 68%). ESI: M+H 971.5. ¹H NMR (600 MHz, DMSO-d6): δ 11.02 (s, 1H), 10.42 (m, 1H), 9.78 (m, 1H), 7.69 (d, J=8.49 Hz, 1H), 7.65 (m, 1H), 7.54 (d, J=8.89 Hz, 1H), 7.41 (d, J=2.01 Hz, 1H), 7.29-7.20 (m, 5H), 7.11 (m, 2H), 7.00 (dd, J=2.01, 8.89 Hz, 1H), 6.93 (s, 2H), 5.53 (s, 2H), 5.02 (dd, J=5.53, 13.14 Hz, 1H)

### EXAMPLE 4: SYNTHESIS OF JMV7506

### Step 1: 8-{4-[2-(2,6-dioxopiperid-3-yl)-1,3-dioxo-2,3-dihydro-1H-isoindol-5-yl] piperazin-1-yl}octanoic Acid

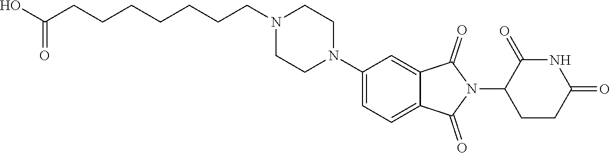 [SMILES: O=C(O)CCCCCCCN1CCN(c2ccc3c(c2)C(=O)N(C2CCC(=O)NC2=O)C3=O)CC1]

The 2-(2,6-dioxopiperidin-3-yl)-5-(piperazin-1-yl)isoindoline-1,3-dione compound (100 mg, 0.29 mmol) (example 2, step 3) is dissolved in acetonitrile (5 ml). 8-bromooctanoic acid (160 mg, 0.73 mmol) and DIEA (0.193 ml, 1.16 mmol) are added and the mixture is stirred at 60° C. for 24 h. The solution is concentrated under reduced pressure. The oil obtained is purified by preparative HPLC. After lyophilization, a yellow powder is obtained with a mass of 101 mg (67% yield). ESI: M+H 485.6.

### Step 2

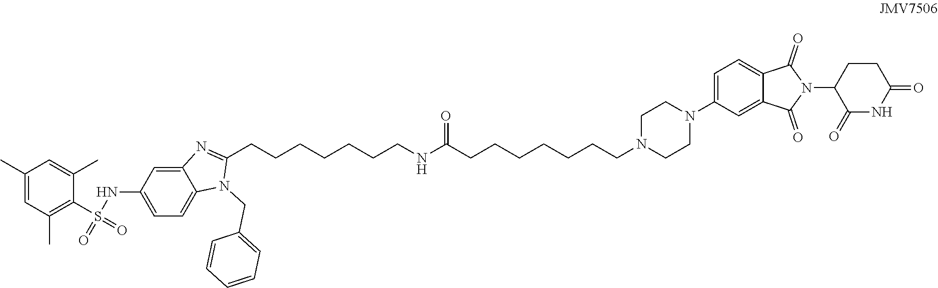 [SMILES: Cc1cc(C)c(S(=O)(=O)Nc2ccc3c(c2)nc(CCCCCCCNC(=O)CCCCCCCN2CCN(c4ccc5c(c4)C(=O)N(C4CCC(=O)NC4=O)C5=O)CC2)n3Cc2ccccc2)c(C)c1]

BOP (52 mg, 0.12 mmol) is added to a solution containing N-[2-(7-aminoheptyl)-1-benzyl-1H-1,3-benzodiazol-5-yl]-2,4,6-trimethylbenzene-1-sulfonamide (41 mg, 0.079 mmol) (example 1, JMV6944), 6-(4-(2-(2,6-dioxopiperidin-3-yl)-1,3-dioxoisoindol-5-yl)piperazin-1-yl)octanoic acid (36 mg, 0.079 mmol) and DIEA (0.039 ml, 0.023 mmol) in DMF (5 ml). The reaction medium is stirred for two hours at room temperature. The solution is concentrated under reduced pressure. The oil obtained is purified by preparative HPLC. After lyophilization, a white powder is obtained with a mass of 45 mg (61% yield). ESI: M+H 985.5.

1H NMR (600 MHz, DMSO-d6): δ 11.02 (s, 1H), 10.42 (m, 1H), 9.78 (m, 1H), 7.69 (d, J=8.49 Hz, 1H), 7.65 (m, 1H), 7.54 (d, J=8.89 Hz, 1H), 7.41 (d, J=2.01 Hz, 1H), 7.29-7.20 (m, 5H), 7.11 (m, 2H), 7.00 (dd, J=2.01, 8.89 Hz, 1H), 6.93 (s, 2H), 5.54 (s, 4H), 5.02 (dd, J=5.53, 13.14 Hz, 1H)

### EXAMPLE 5: SYNTHESIS OF JMV7965

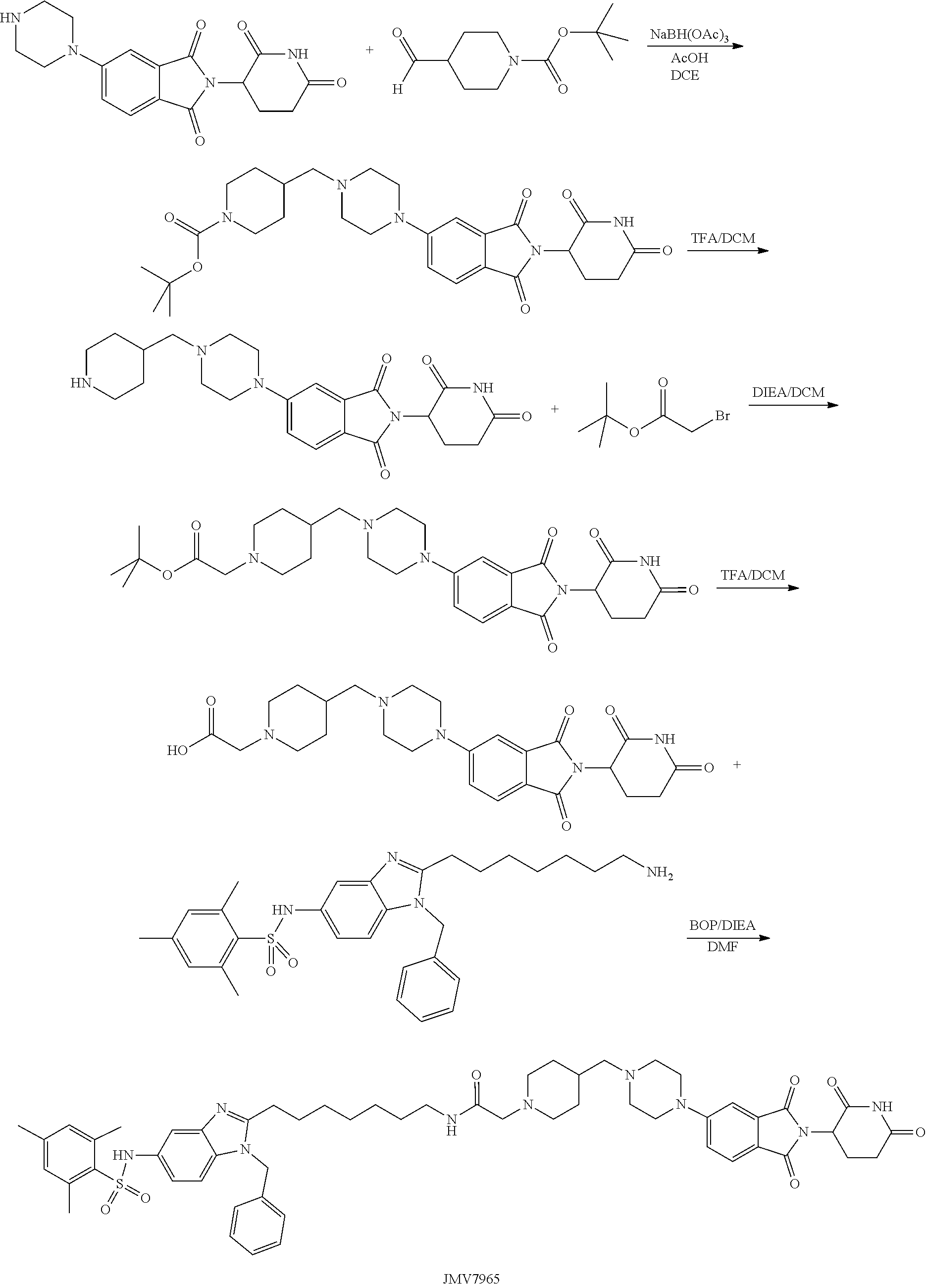

### Step 1: tert-butyl 4-({4-[2-(2,6-dioxopiperidin-3-yl)-1,3-dioxo-2,3-dihydro-1H-isoindol-5-yl]piperazin-1-yl}methyl)piperidine-1-carboxylate

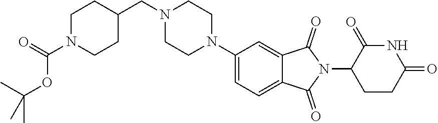 [SMILES: CC(C)(C)OC(=O)N1CCC(CN2CCN(c3ccc4c(c3)C(=O)N(C3CCC(=O)NC3=O)C4=O)CC2)CC1]

3 ml of MeOH is added to a solution containing 2-(2,6-dioxopiperidin-3-yl)-5-(piperazin-1-yl)isoindoline-1,3-dione (100 mg, 0.29 mmol) (example 2, step 3) and tert-butyl 4-formylpiperidin-1-carboxylate (112 mg, 0.53 mmol) in DCE. The reaction medium is stirred at room temperature for 30 min. Add the sodium triacetoxyborohydride by portions and stir the reaction medium at room temperature for 18 h. Concentrate the reaction medium and carry out preparative HPLC. After lyophilization, a yellow powder is obtained with a mass of 85 mg (yield 53%). ESI: M+H 540.2.

### Step 2: 2-(2,6-dioxopiperidin-3-yl)-5-{4-[(piperidin-4-yl)methyl]piperazin-1-yl}-2,3-dihydro-1H-isoindole-1,3-dione

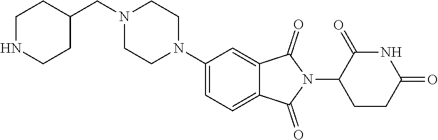 [SMILES: O=C1CCC(N2C(=O)c3ccc(N4CCN(CC5CCNCC5)CC4)cc3C2=O)C(=O)N1]

The tert-butyl 4-({4-[2-(2,6-dioxopiperidin-3-yl)-1,3-dioxo-2,3-dihydro-1H-isoind-5-yl]piperazin-1-yl}methyl)piperidine-1-carboxylate compound (100 mg, 0.19 mmol) is dissolved in DCM (50 ml). TFA (5 ml) is added drop-by-drop to the reaction medium and stirred at room temperature for 5 h. The solution is concentrated under reduced pressure. The oil obtained (75 mg, yield 92%) is used as is in step 3. ESI: M+FI 440.3.

### Step 3: tert-butyl 2-[4-({4-[2-(2,6-dioxopiperidin-3-yl)-1,3-dioxo-2,3-dihydro-1H-isoind-5-yl]piperazin-1-yl}methyl)piperidin-1-yl]acetate

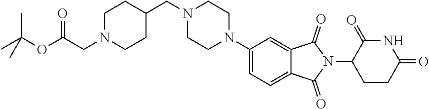 [SMILES: CC(C)(C)OC(=O)CN1CCC(CN2CCN(c3ccc4c(c3)C(=O)N(C3CCC(=O)NC3=O)C4=O)CC2)CC1]

The compound 2-(2,6-dioxopiperidin-3-yl)-5-{4-[(piperidin-4-yl)methyl]piperazin-1-yl}-2,3-dihydro-1H-isoindole-1,3-dione (128 mg, 0.29 mmol) is dissolved in DCM in the presence of DIEA (0.14 ml, 0.88 mmol). Add tert-butyl bromoacetate (0.043 ml, 0.29 mmol) and stir at room temperature for 18 h. Concentrate the reaction medium and carry out preparative HPLC. After lyophilization, a yellow powder is obtained with a mass of 85 mg (yield 53%). ESI: M+H 554.4.

### Step 4: 2-[4-({4-[2-(2,6-dioxopiperidin-3-yl)-1,3-dioxo-2,3-dihydro-1H-isoind-5-yl]piperazin-1-yl}methyl)piperidin-1-yl]acetic Acid

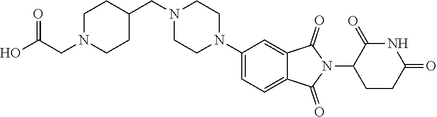 [SMILES: O=C(O)CN1CCC(CN2CCN(c3ccc4c(c3)C(=O)N(C3CCC(=O)NC3=O)C4=O)CC2)CC1]

The tert-butyl 2-[4-({4-[2-(2,6-dioxopiperidin-3-yl)-1,3-dioxo-2,3-dihydro-1H-isoind-5-yl]piperazin-1-yl}methyl)piperidin-1-yl] acetate compound (83 mg, 0.19 mmol) is dissolved in DCM (25 ml). TFA (5 ml) is added drop-by-drop to the reaction medium and stirred at room temperature for 5 h. The solution is concentrated under reduced pressure. The oil obtained (70 mg, yield 93%) is used as is in step 3. ESI: M+H 498.3.

### Step 5

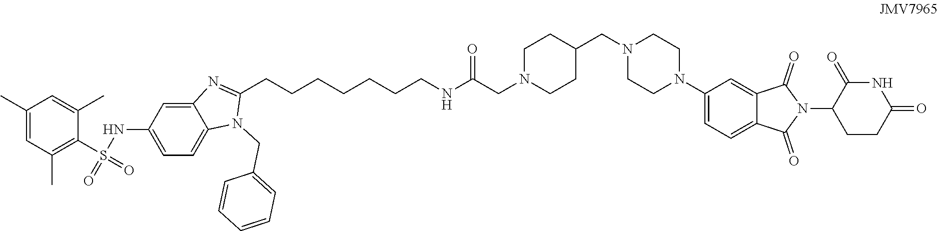 [SMILES: Cc1cc(C)c(S(=O)(=O)Nc2ccc3c(c2)nc(CCCCCCCNC(=O)CN2CCC(CN4CCN(c5ccc6c(c5)C(=O)N(C5CCC(=O)NC5=O)C6=O)CC4)CC2)n3Cc2ccccc2)c(C)c1]

BOP (27 mg, 0.0603 mmol) is added to a solution containing N-[2-(7-aminoheptyl)-1-benzyl-1H-1,3-benzodiazol-5-yl]-2,4,6-trimethylbenzene-1-sulfonamide (21 mg, 0.0402 mmol) (example 1, JMV6944), 2-[4-({4-[2-(2,6-dioxopiperidin-3-yl)-1,3-dioxo-2,3-dihydro-1H-isoindol-5-yl]piperazin-1-yl}methyl)piperidin-1-yl] acetic acid (20 mg, 0.0402 mmol) and DIEA (0.020 ml, 0.12 mmol) in DMF (5 ml). The reaction medium is stirred for two hours at room temperature. The solution is concentrated under reduced pressure. The oil obtained is purified by preparative HPLC. After lyophilization, a yellow powder is obtained with a mass of 25 mg (62% yield). ESI: M+H 998.3.

### EXAMPLE 6: SYNTHESIS OF JMV7605

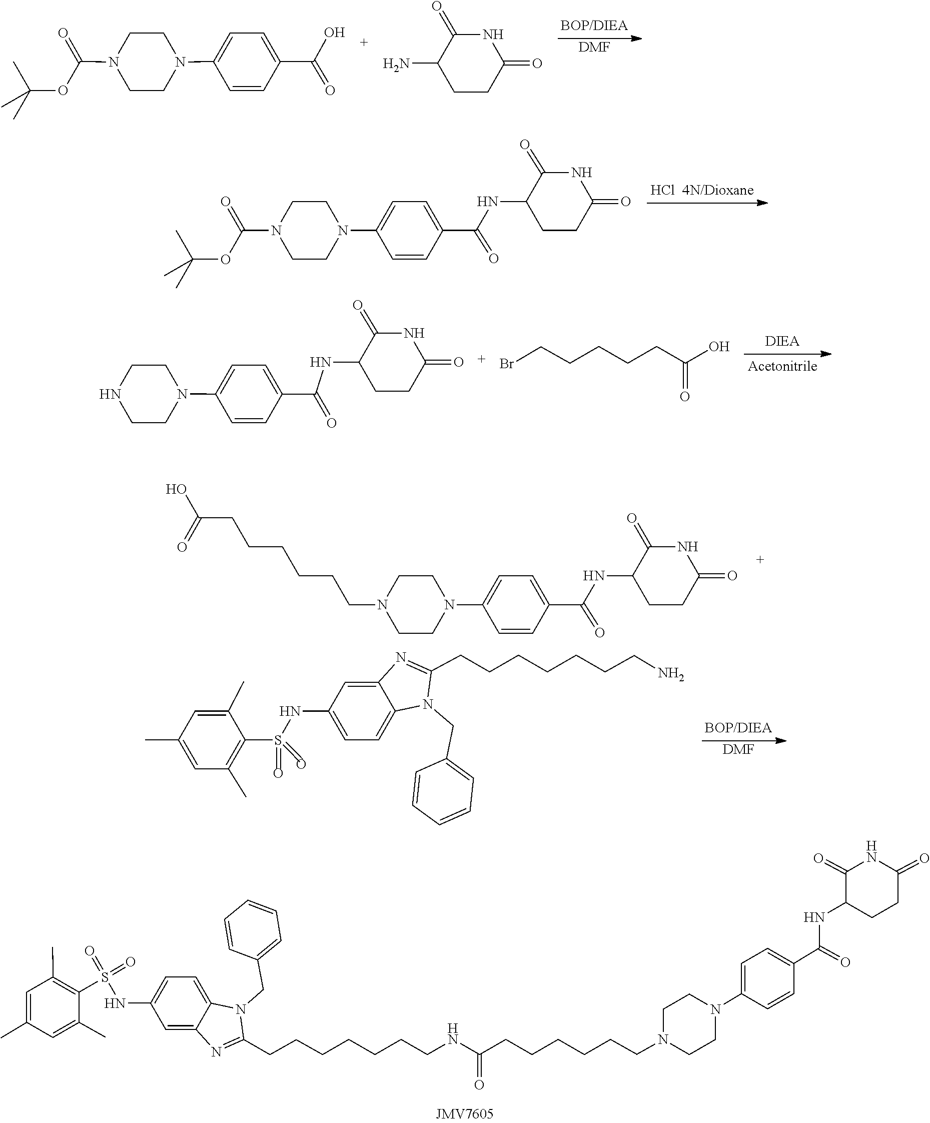

### Step 1: tert-butyl 4-{4-[(2,6-dioxopiperidin-3-yl)carbamoyl]phenyl}piperazine-1-carboxylate

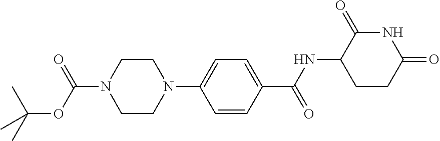 [SMILES: CC(C)(C)OC(=O)N1CCN(c2ccc(C(=O)NC3CCC(=O)NC3=O)cc2)CC1]

BOP (1.11 g, 2.53 mmol) is added to a solution containing 4-[4-(tert-butoxycarbonyl)piperazino]benzoic acid (0.775 g, 2.53 mmol), 3-aminopiperidine-2,6-dione HCl (0.50 g, 3.03 mmol) and DIEA (1.25 ml, 7.59 mmol) in DMF (50 ml).

The reaction medium is stirred for two hours at room temperature. Add water to the reaction medium and extract with ethyl acetate. Successively wash the organic phase with 1N HCl, saturated NaHCO₃ and saturated NaCl. Dry the organic phase with MgSO4, filter and concentrate under reduced pressure. A white powder is obtained with a mass of 0.4 g (38% yield). ESI: M+H 417.3.

### Step 2: N-(2,6-dioxopiperidin-3-yl)-4-(piperazin-1-yl)benzamide

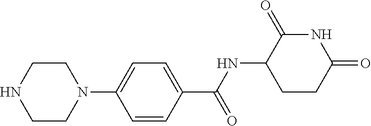 [SMILES: O=C1CCC(NC(=O)c2ccc(N3CCNCC3)cc2)C(=O)N1]

The tert-butyl 4-{4-[(2,6-dioxopiperidin-3-yl)carbamoyl]phenyl}piperazine-1-carboxylate (0.4 g, 0.96 mmol) compound is dissolved in a 4N solution of HCl in dioxane (6 ml) and the reaction medium is stirred at room temperature for 2 hours and then concentrated and triturated with ether. The solid obtained in the form of a white powder with a mass of 0.285 mg (94% yield).

The reaction medium is stirred for two hours at room temperature. The solution is concentrated under reduced pressure. The oil obtained is purified by preparative HPLC. After lyophilization, a white powder is obtained with a mass of 45 mg (52% yield). ESI: M+H 317.3.

### Step 3: 7-(4-{4-[(2,6-dioxopiperidin-3-yl)carbamoyl]phenyl}piperazin-1-yl)heptanoic Acid

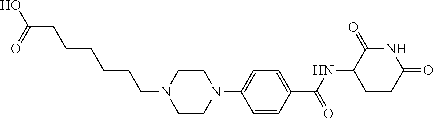 [SMILES: O=C(O)CCCCCCN1CCN(c2ccc(C(=O)NC3CCC(=O)NC3=O)cc2)CC1]

The N-(2,6-dioxopiperidin-3-yl)-4-(piperazin-1-yl)benzamide compound (50 mg, 0.15 mmol) is dissolved in DMF (5 ml). 7-bromoheptanoic acid (66 mg, 0.31 mmol) and DIEA (0.078 ml, 0.47 mmol) are added and the mixture is stirred at 100° C. for 24 h. The solution is concentrated under reduced pressure. The oil obtained is purified by preparative HPLC. After lyophilization, a yellow powder is obtained with a mass of 38 mg (55% yield). ESI: M+H 445.1.

### Step 4

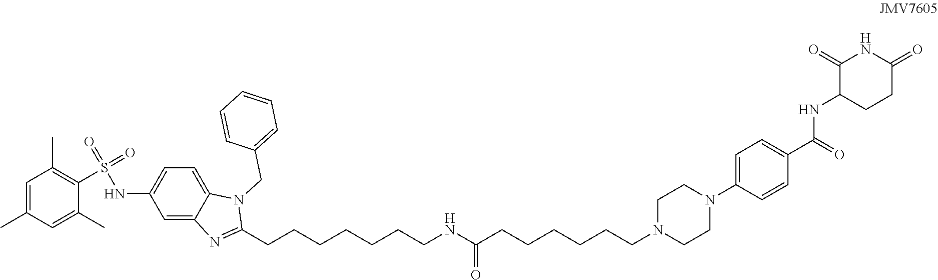 [SMILES: Cc1cc(C)c(S(=O)(=O)Nc2ccc3c(c2)nc(CCCCCCCNC(=O)CCCCCCN2CCN(c4ccc(C(=O)NC5CCC(=O)NC5=O)cc4)CC2)n3Cc2ccccc2)c(C)c1]

BOP (44 mg, 0.101 mmol) is added to a solution containing N-[2-(7-aminoheptyl)-1-benzyl-1H-1,3-benzodiazol-5-yl]-2,4,6-trimethylbenzene-1-sulfonamide (35 mg, 0.067 mmol) (example 1, JMV6944), 7-(4-{4-[(2,6-dioxopiperidin-3-yl)carbamoyl]phenyl}piperazin-1-yl)heptanoic acid (30 mg, 0.067 mmol) and DIEA (0.033 ml, 0.20 mmol) in DMF (5 ml). The reaction medium is stirred for two hours at room temperature. The solution is concentrated under reduced pressure. The oil obtained is purified by preparative HPLC. After lyophilization, a white powder is obtained with a mass of 41 mg (65% yield). ESI: M+H 945.8.

### EXAMPLE 7: SYNTHESIS OF JMV7159 (COMPARATIVE EXAMPLE)

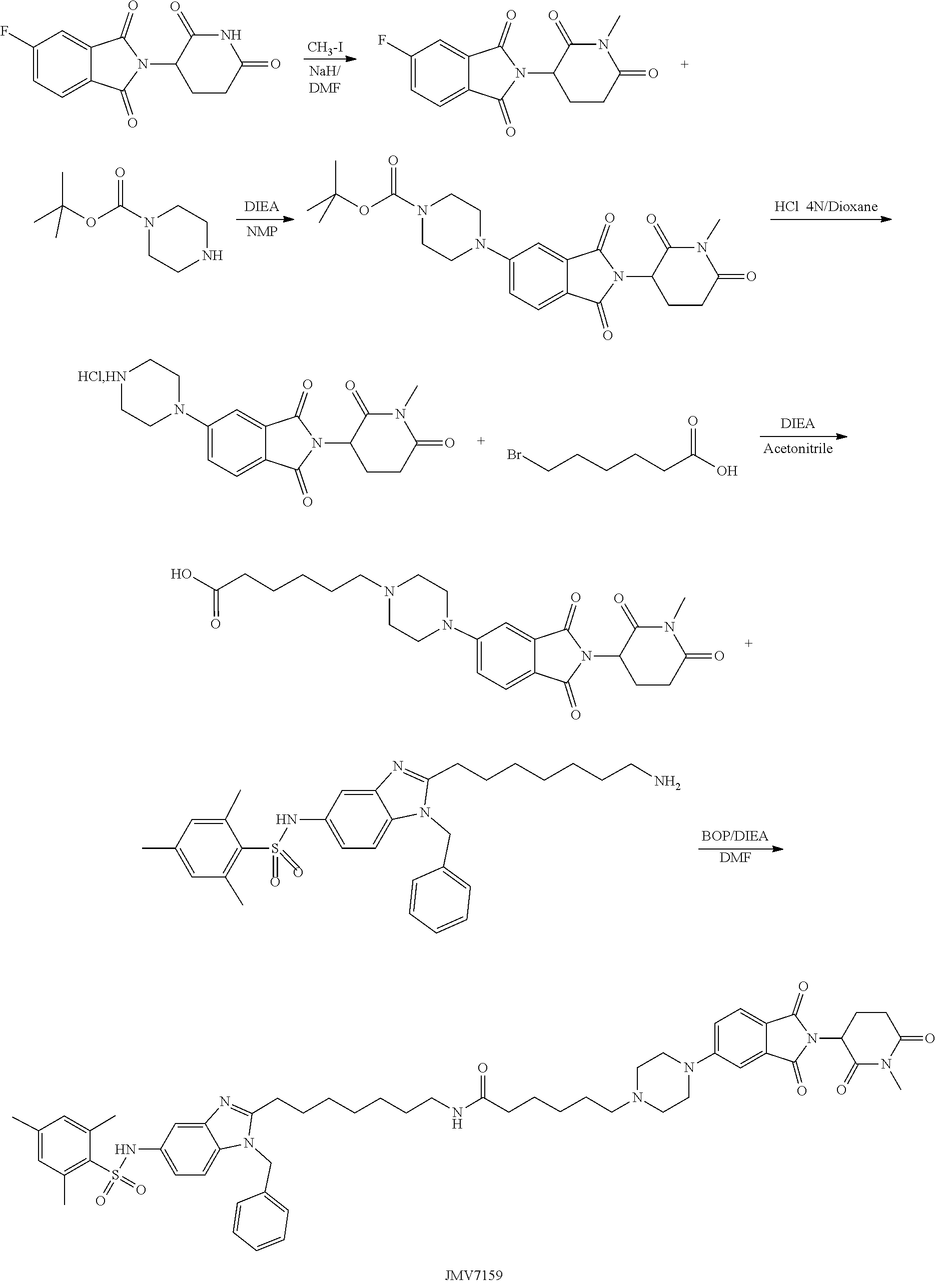

### Step 1: 5-fluoro-2-(1-methoxy-2,6-dioxopiperidin-3-yl)-2,3-dihydro-1H-isoindole-1,3-dione

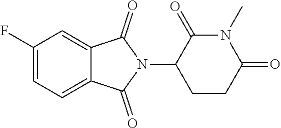 [SMILES: CN1C(=O)CCC(N2C(=O)c3ccc(F)cc3C2=O)C1=O]

The compound 2-(2,6-dioxopiperidin-3-yl)-5-fluoroisoindoline-1,3-dione (250 mg, 0.90 mmol) is dissolved in anhydrous DMF (5 ml) and the reaction medium is stirred and brought to 0° C. Add NaH by portions and stir for 20 minutes. Add methyl iodide and stir for 2 hours. Stop the reaction with an NH4Cl solution. Extract with ethyl acetate and wash the organic phase twice with saturated NaCl. Dry over MgS04, filter and concentrate under reduced pressure. The compound 5-fluoro-2-(1-methyl-2,6-dioxopiperidin-3-yl)-2,3-dihydro-1H-isoindole-1,3-dione is obtained in the form of a white powder with a mass of 253 mg (96% yield). ESI: M+H 291.1.

### Step 2: tert-butyl 4-[2-(1-methyl-2,6-dioxopiperidin-3-yl)-1,3-dioxo-2,3-dihydro-1H-isoindol-5-yl]piperazine-1-carboxylate

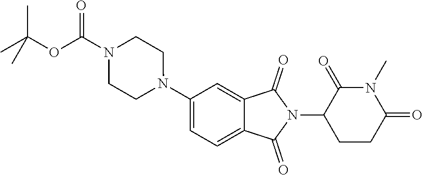 [SMILES: CN1C(=O)CCC(N2C(=O)c3ccc(N4CCN(C(=O)OC(C)(C)C)CC4)cc3C2=O)C1=O]

The compound 5-fluoro-2-(1-methyl-2,6-dioxopiperidin-3-yl)-2,3-dihydro-1H-isoindole-1,3-dione (250 mg, 0.86 mmol) is dissolved in NMP (4 ml) at room temperature. DIEA (0.42 ml, 2.58 mmol) and t-butyl 1-piperazine-carboxylate (176 mg, 0.94 mmol) are added and the mixture is stirred at 140° C. for 24 h. The solution is diluted in water (100 ml). Extract twice with ethyl acetate and the organic phase is washed with saturated NaCl and dried over magnesium sulfate. After evaporation, the oil obtained is purified on silica gel with a petroleum ether/ethyl acetate eluent (3/1). Tert-butyl 4-[2-(1-methyl-2,6-dioxopiperid-3-yl)-1,3-dioxo-2,3-dihydro-1H-isoindol-5-yl]piperazine-1-carboxylate is obtained in the form of a yellow solid with a mass of 338 mg (86% yield). ESI: M+H 457.3.

### Step 3: 2-(1-methyl-2,6-dioxopiperidin-3-yl)-5-(piperazin-1-yl)-2,3-dihydro-1H-isoindole-1,3-dione

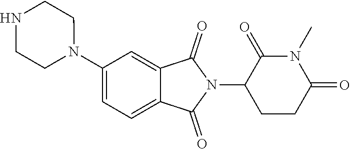 [SMILES: CN1C(=O)CCC(N2C(=O)c3ccc(N4CCNCC4)cc3C2=O)C1=O]

The tert-butyl 4-[2-(1-methyl-2,6-dioxopiperidin-3-yl)-1,3-dioxo-2,3-dihydro-1H-isoindol-5-yl]piperazine-1-carboxylate (250 mg, 0.54 mmol) compound is dissolved in a 4N solution of HCl in dioxane (4 ml) and the reaction medium is stirred at room temperature for 2 hours and then concentrated and triturated with ether. The solid obtained in the form of a yellow powder with a mass of 175 mg (90% yield). ESI: M+H 357.3.

### Step 4: 6-{4-[2-(1-methyl-2,6-dioxopiperidin-3-yl)-1,3-dioxo-2,3-dihydro-1H-isoindol-5-yl]piperazin-1-yl}hexanoic Acid

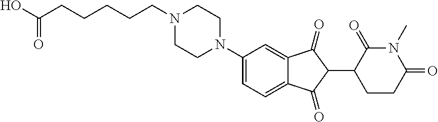 [SMILES: CN1C(=O)CCC(C2C(=O)c3ccc(N4CCN(CCCCCC(=O)O)CC4)cc3C2=O)C1=O]

The compound 2-(1-methyl-2,6-dioxopiperidin-3-yl)-5-(piperazin-1-yl)-2,3-dihydro-1H-isoindole-1,3-dione (100 mg, 0.28 mmol) is dissolved in acetonitrile (5 ml). 6-bromohexanoic acid (136 mg, 0.70 mmol) and DIEA (0.139 ml, 0.84 mmol) are added and the mixture is stirred at 60° C. for 24 h. The solution is concentrated under reduced pressure. The oil obtained is purified by preparative HPLC. After lyophilization, a yellow powder is obtained with a mass of 85 mg (65% yield). ESI: M+H 471.3.

### Step 5

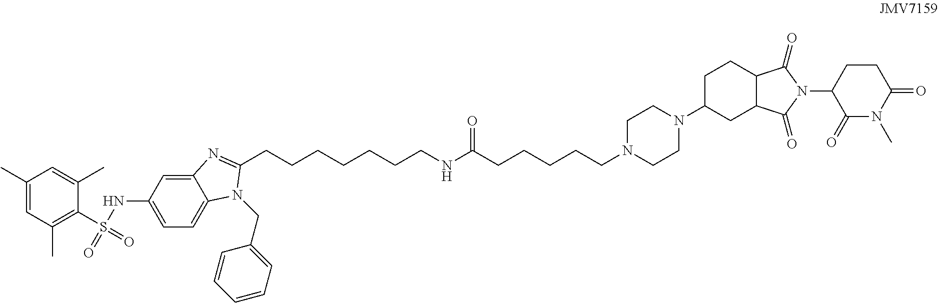 [SMILES: Cc1cc(C)c(S(=O)(=O)Nc2ccc3c(c2)nc(CCCCCCCNC(=O)CCCCCN2CCN(C4CCC5C(=O)N(C6CCC(=O)N(C)C6=O)C(=O)C5C4)CC2)n3Cc2ccccc2)c(C)c1]

BOP (36 mg, 0.082 mmol) is added to a solution containing N-[2-(7-aminoheptyl)-1-benzyl-1H-1,3-benzodiazol-5-yl]-2,4,6-trimethylbenzene-1-sulfonamide (28 mg, 0.055 mmol) (example 1, JMV6944), 6-{4-[2-(1-methyl-2,6-dioxopiperidin-3-yl)-1,3-dioxo-2,3-dihydro-1H-isoindol-5-yl]piperazine-1-yljhexanoic acid (26 mg, 0.055 mmol) and DIEA (0.165 ml, 0.165 mmol) in DMF (5 ml). The reaction medium is stirred for two hours at room temperature. The solution is concentrated under reduced pressure. The oil obtained is purified by preparative HPLC. After lyophilization, a white powder is obtained with a mass of 30 mg (56% yield). ESI: M+H 971.6.

### EXAMPLE 8: BIOCHEMISTRY AND CRYSTALLOGRAPHY

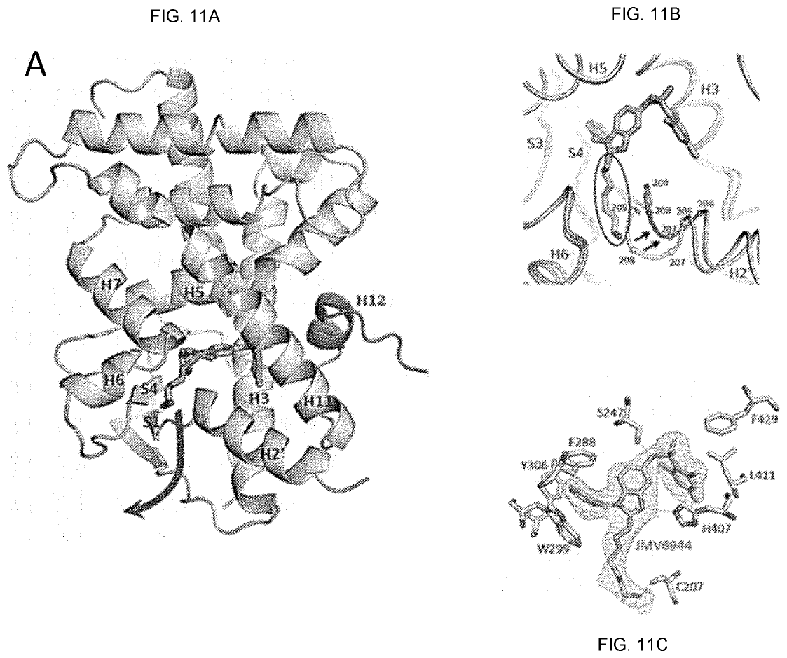

The human PXR receptor ligand binding domain (hPXR-LBD, residues 130-434) was produced in the form of a recombinant protein in E. coli BL21-DE3 bacteria. The protein was purified on an affinity column then by size exclusion chromatography. After concentration, hPXR-LBD was crystallized in the presence of the JMV6944 ligand. The structure of the hPXR-LBD/JMV6944 complex was determined by radiocrystallography by the molecular replacement method, then rebuilt and refined on the basis of the electron density (diffraction data collected at the ERF synchrotron, Grenoble). The structure is shown in FIG. 11. In A, the entire structure of the complex shows the binding mode of JMV6944. The originality of JMV6944 lies in the position of the extension added to the parent molecule JMV6845 and its pathway for exiting the protein domain. Unlike the PROTACs known for the other nuclear receptors which are all based on the modification of an antagonist ligand, the extension grafted on the JMV6845 agonist does not extend toward the H12 helix but instead points in the opposite direction between the H2′, H6 and H7 helices and the S1 strand in order to finally reach the external surface of the LBD. The presence of the alkyl/NH arm (surrounded by B) induces a conformational change of the H2′ end which allows this ligand to be extracted from the binding pocket and to interact specifically with a surface residue, C207 (C). Within the binding pocket of the ligand, JMV6944 also establishes hydrogen bonds with H407 and S247, as well as hydrophobic interactions with L411 and F428, as well as with the residues of the “π-trap” region (F288, W299, Y306).

### EXAMPLE 9: BIOLOGICAL RESULTS

9.1 Measurement of prePROTAC/PXR Affinity

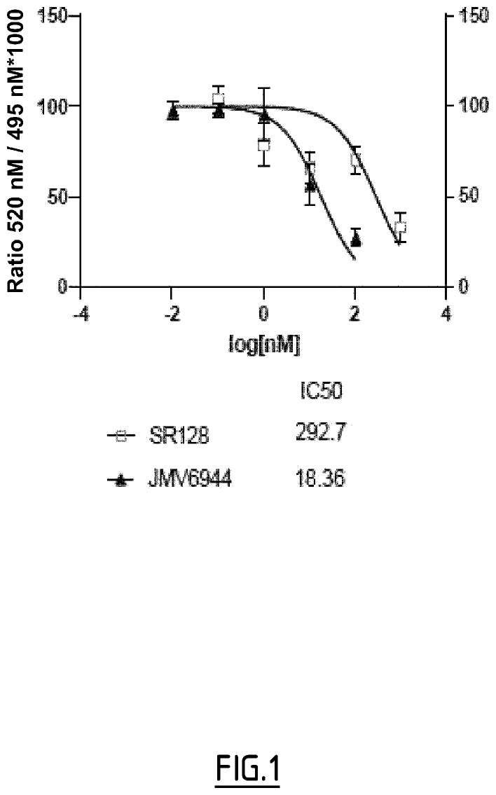

The binding affinity between the JMV6944 (prePROTAC) molecule and the PXR ligand binding domain (LBD) was quantified by FRET by virtue of the LanthaScreen TR-FRET PXR competitive binding assay kit (Invitrogen). The molecules were incubated for 1:30 hours at room temperature with the PXR LBD in the presence of a fluorescent reference ligand. The displacement of the fluorescent ligand caused by the prePROTAC or the PXR SR12813 ligand was measured by reading emissions at 520 nm and 495 nm after excitation at 337 nm on a PHERA-Star apparatus (BMG LABTECH). The results are illustrated in FIG. 1, which shows that the molecule JMV6944 is a PXR ligand with an affinity of 18.38 nm.

### 9.2 Measurement of the Effects of the PROTACs on the Transcriptional Activity of PXR (Reporter Gene)

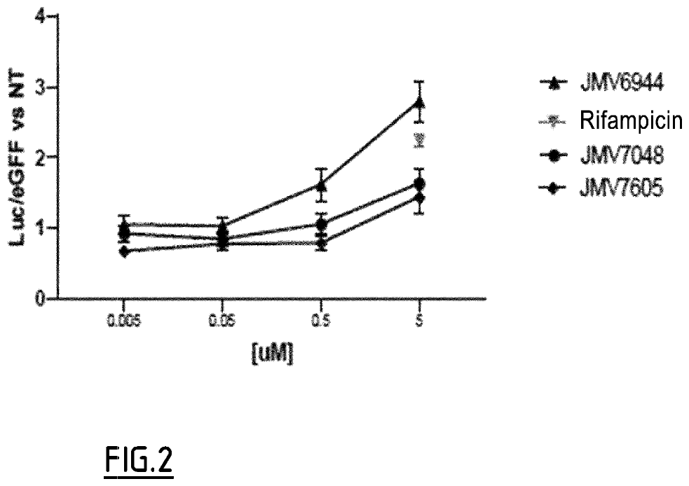

Treatment of LS174T cells stably transfected with an expression vector encoding the PXR protein, a Luciferase reporter gene placed under the control of the CYP3A4 promoter (PXR target gene) and an expression cassette encoding for the GFP protein placed under the control of the CMV promoter for the normalization of the signals. The cells were treated for 48 h with 5 μM of molecules JMV6944 (prePROTACs), the PROTACs JMV7048 and JMV7605, as well as rifampicin (5 μM, PXR ligand). At the end of the treatments, the transcriptional activity of PXR is measured by the ratio of the luciferase/GFP signals measured on a PHERA-Star apparatus (BMG LABTECH). FIG. 2 shows that only prePROTAC JMV6944 and rifampicin are capable of activating the transcriptional activity of PXR.

9.3 Measuring the Effects of the PROTACs on the Transcriptional Activity of PXR (CYP3A4 mRNA Expression)

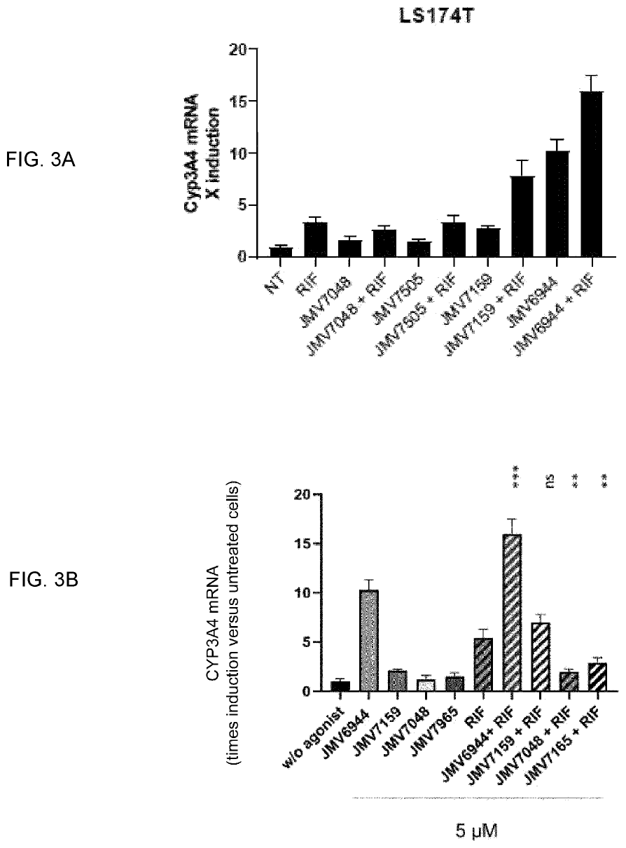

The LS174T cells were treated for 48 h with 5 μM of molecules JMV6944 (prePROTACs), JMV7048, JMV7505, or JMV5159 (inactive equivalent of JMV7048 following the addition of a methyl group on the CNBR ligase ubiquitin ligand) in the presence or absence of rifampicin (PXR ligand) at 5 μM final. After lysis of the cells and purification of the total RNAs (Qiagen RNAeasy), the complementary DNAs were prepared (SuperScript II in the presence of random primers of 6 nucleotides, Invitrogen). The expression of CYP3A4 mRNA and RPLO and actin housekeeping genes was measured by RT-qPCR on an LC480 apparatus (Roche) in the presence of SyberGreen (Millipore). The relative expression levels were calculated according to the RQ=Relative quantification=2-ΔΔCt method, the untreated cells serving as a calibrator set to 1. FIGS. 3A and 3B show that if the prePROTAC and the inactivated PROTAC (JMV7159) have an additive effect on the expression of CYP3A4 mRNA, the PROTACs JMV7048 and JMV7965 significantly reduce the induction of CYP3A4 mediated by rifampicin.

### 9.4 Measurement of the Effects of the PROTACs on the Transcriptional Activity of PXR (Expression of CYP3A4)

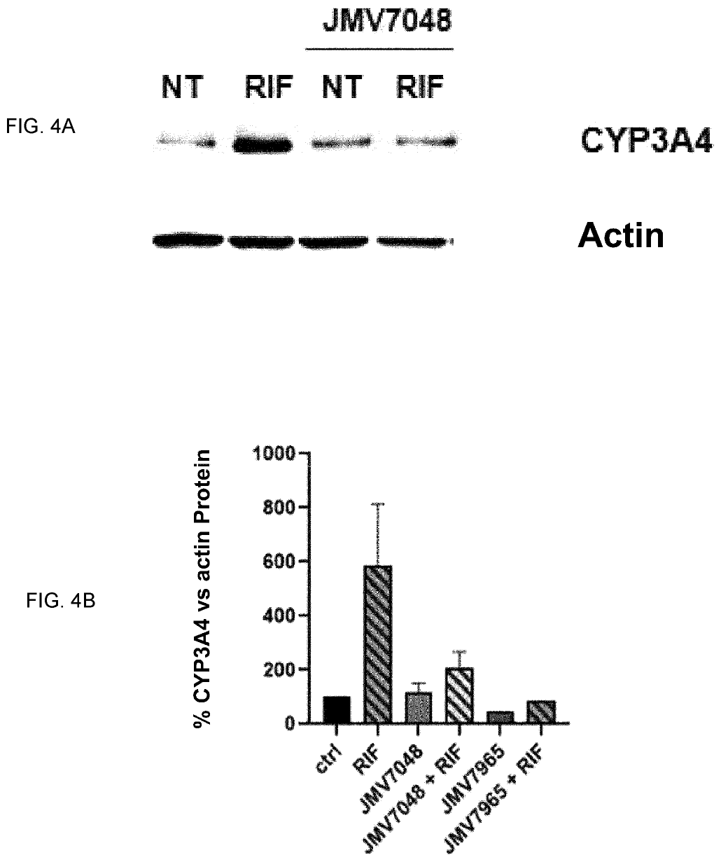

The LS174T cells were treated for 48 h with 5 mM JMV7048 in the presence or absence of rifampicin (PXR ligand) at 5 mM final. After lysis of the cells (RIPA+antiproteases), the proteins were purified and assayed before being deposited (90 μg) on 10% SDS-PAGE gel. After the migration onto the gel, they were transferred to a nitrocellulose membrane (GE Healthcare) before being revealed with antibodies directed against CYP3A4 (sc-53850, Santa Cruz) and beta-actin (A5441, Sigma or Ab-253283, AbCAm) and then secondary antibodies coupled to peroxidase (anti-mouse HRP, Santa Cruz). The intensities of the signals were measured by a camera (BioRad MP Touch). FIGS. 4A and 4B show that the PROTACs JMV7048 and JMV7965 reduce the induction of the CYP3A4 enzyme mediated by rifampicin.

### 9.5 Measurement of the Effects of the PROTACs on Cell Viability

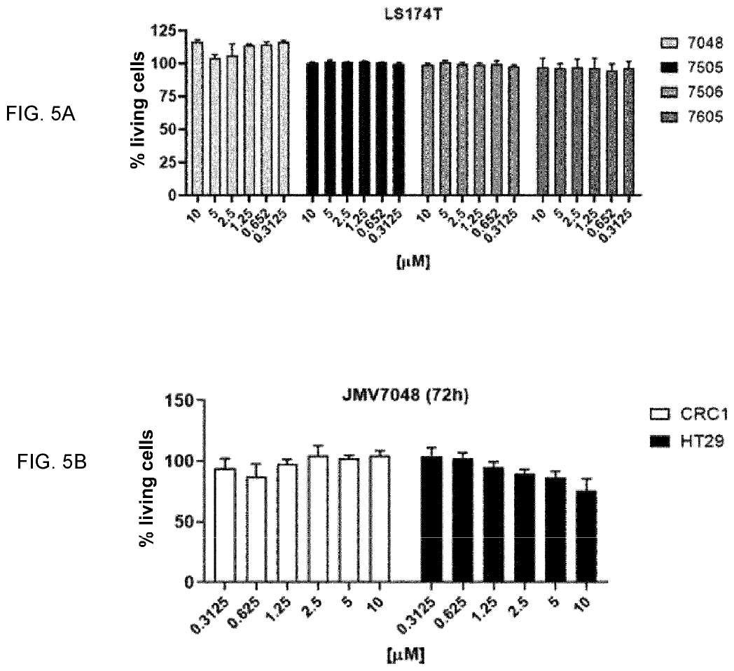

The effect of PROTACs on cell viability has been tested on various lines CRC1, HT29 and LS174T. The cells were incubated in the presence of increasing concentrations of molecules for 72 h before being fixed and marked by Sulforhodamine B (Sigma). After washing and lysis of the cells, the incorporated colorant released by the cells is directly proportional to the cell biomass. It is measured at 565 nM by a 96-well plate spectrophotometer (Tecan). The signal obtained for the untreated cells is set at 100%. FIG. 5A illustrates the absence of toxicity of the PROTACs JMV7048, JMV7505 and JMV7605 on the LS174T line. In FIG. 5B, it can be seen that the PROTAC JMV7048 does not affect the viability of HT29 cells or of the CRC1 protoculture (derived from a patient with colon cancer.

### 9.6 Measurement of the Effects of the PROTACs on PXR Degradation in the LS174T Cells by In Vitro Western Blotting

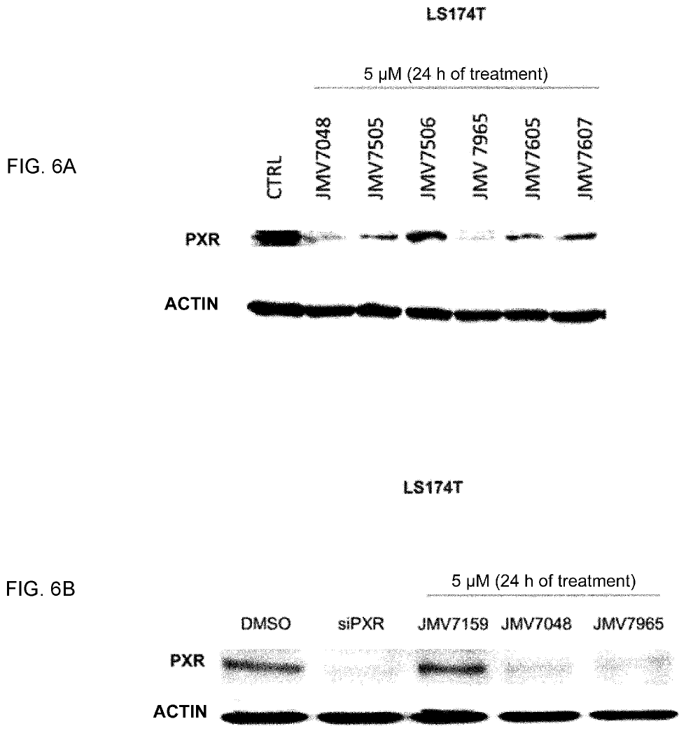

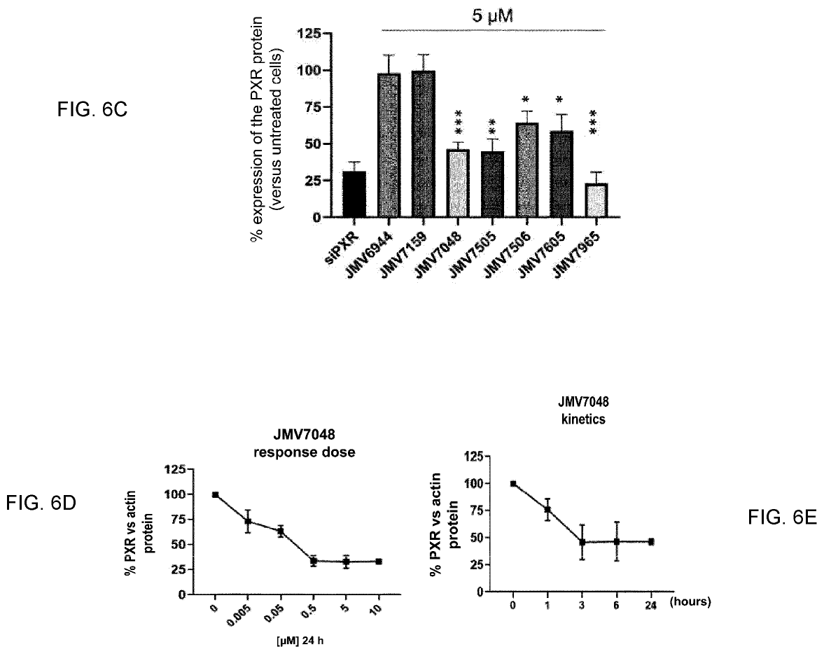

The effect of the PROTACs on the expression level of the PXR protein was studied by western blotting. The LS174T cells were transplanted in the absence or presence of 50 nM of an siRNA targeting PXR (siPXR: NR112 Silencer, Thermofischer) or treated by the PROTACs. After lysis of the cells (RIPA+antiproteases), the proteins were purified and assayed before being deposited (9C\hat{ }g) on 10% SDS-PAGE gel. After migration onto the gel, they were transferred to a nitrocellulose membrane (GE Healthcare) before being revealed with antibodies directed against PXR (sc-48340, Santa Cruz), GAPDH (sc-32233, Santa Cruz) and beta-actin (A5441, Sigma or Ab-253283, AbCAm) and then secondary antibodies coupled to peroxidase (anti-mouse HRP, Santa Cruz). The intensities of the signals were measured by a camera (BioRad MP Touch). FIGS. 6A-C show that after 24 h of treatment at 5 μM, the PROTACs JMV7048, JMV7505, JMV7506, JMV7605 and JMV7965 significantly reduce the expression level of the PXR protein, unlike an inactive mutant of JMV7048 (i.e. JMV7159). FIGS. 6D and 6E illustrate the effect of JMV7048 on the expression level of PXR based on the treatment time (maximum effect reached after 3 h of treatment) and the concentration used (decrease dependent on the dose, with a maximum effect observed from 500 nM).

### 9.7 Measurement of the Effects of the PROTACs on PXR Degradation in the HEPG2 and ASPC1 Cells by In Vitro Western Blotting

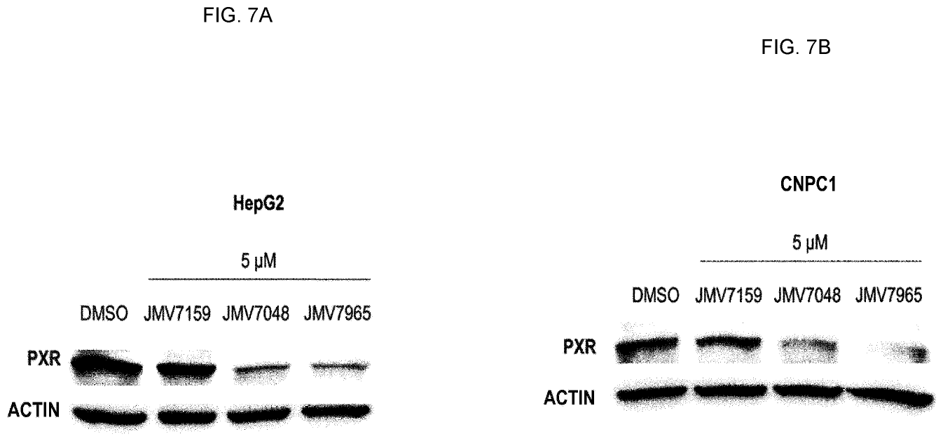

The effect of the PROTACs (5 mM, 24 h of treatment) on the expression level of the PXR protein in HepG2 (Hepatocellular Carcinoma, ATCC #HB-8065™) or ASPC1 (Human Pancreatic Cancer Cell Line, ATCC #CRL-1682) cells was studied by western blotting. After lysis of the cells (RIPA+antiproteases), the proteins were purified and assayed before being deposited (90 μg) on 10% SDS-PAGE gel. After migration onto the gel, they were transferred to a nitrocellulose membrane (GE Healthcare) before being revealed with antibodies directed against PXR (sc-48340, Santa Cruz), GAPDH (sc-32233, Santa Cruz) and beta-actin (A5441, Sigma or Ab-253283, AbCAm) and then secondary antibodies coupled to peroxidase (anti-mouse HRP, Santa Cruz). FIGS. 7A and 7B illustrate the effect of the PROTACs JMV7048 and JMV7965 on the expression level of PXR in hepatic cancer cells (7A) or pancreatic cancer cells (7B)

### 9.8 Importance of the Proteasome Pathway on the Effect of the PROTACs on PXR Degradation.

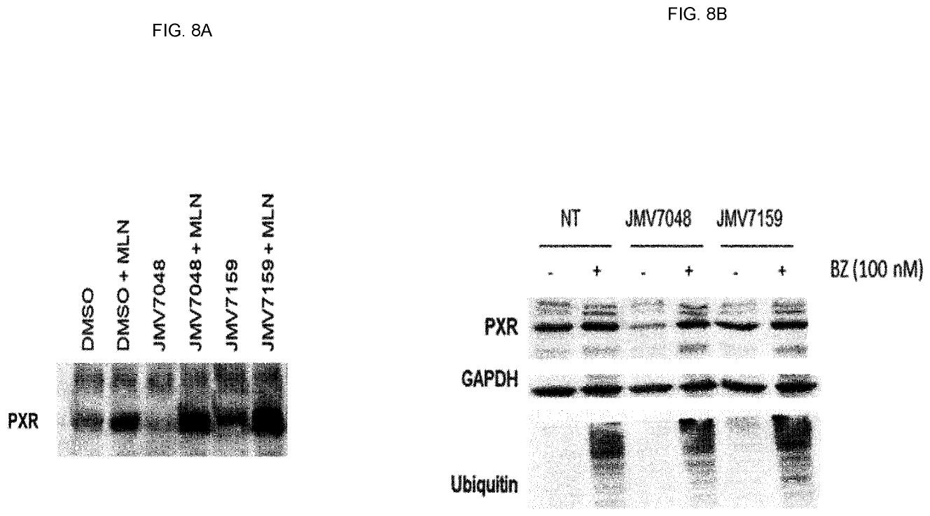

The involvement of the proteasome pathway in the effects of PROTACs on the expression level of the PXR protein was studied by western blotting. The LS174T cells were treated for 24 hours with JMV7048 in the presence or absence of the CNBR ubiquitin ligase (MLN4924) or the proteasome inhibitor (Bortezomib). FIGS. 8A and 8B confirm the importance of the proteasome pathway on the decrease in the expression level of the PXR protein induced by the PROTAC JMV7048: the decrease in the expression level of PXR induced by JMV7048 is reverted by the CRBN ubiquitin ligase inhibitors (MLN4924, FIG. 8A) or by an inhibitor of the 26S proteasome (Bortezomib, Bz; FIG. 8B), whereas the mutant of the JMV7048 (i.e. JMV7159, not allowing the recruitment of the CNBR) does not cause a decrease in the expression level of PXR.

### 9.9 Measurement of the Effects of the PROTACs on PXR Degradation by In Vivo Western Blotting

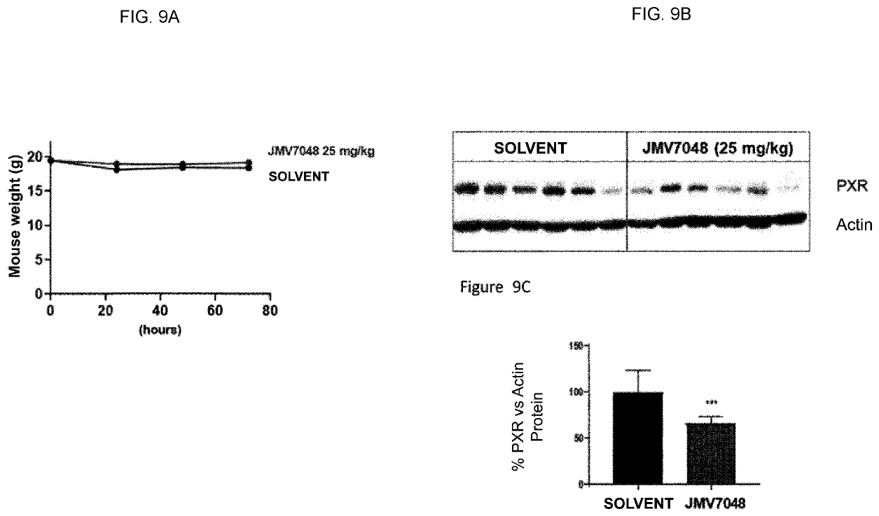

The effect of the PROTACs on the expression level of the PXR protein was studied in vivo by western blotting from xenografts of LS174T cells in SCID mice. Once the tumors reached 100 mm3, 10 mice were treated by I.V. with 5% EtOH solvent, 20% solutol in D5W or PROTACs (25 mg/kg) every 24 hours for 4 days. The mice were weighed every day. Four hours after the last treatment, the tumors were resected before being lysed in an RIPA buffer by virtue of ceramic beads (Lysing matrix D, MP-Bio) with a Fast-Prep 24 (MP-Bio) apparatus. The proteins were purified and assayed before being deposited (90 μg) on 10% SDS-PAGE gel. After migration onto the gel, they were transferred to a nitrocellulose membrane (GE Healthcare) before being revealed with antibodies directed against PXR (sc-48340, Santa Cruz), GAPDH (sc-32233, Santa Cruz) and beta-actin (A5441, Sigma or Ab-253283, AbCAm) and then secondary antibodies coupled to peroxidase (anti-mouse HRP, Santa Cruz). The intensities of the signals were measured by a camera (Biorad MP Touch). It can be seen in FIG. 9A that a treatment at 25 mk/kb for 4 days does not significantly modify the weight of the mice. FIGS. 9B and 9C confirm that this treatment is capable of inducing a significant drop in the expression level of the PXR protein within the tumors.

### 9.10 Measurement of the Effects of the PROTACs on the Self-Renewal and the Chemoresistance of Colic Cancer Stem Cells

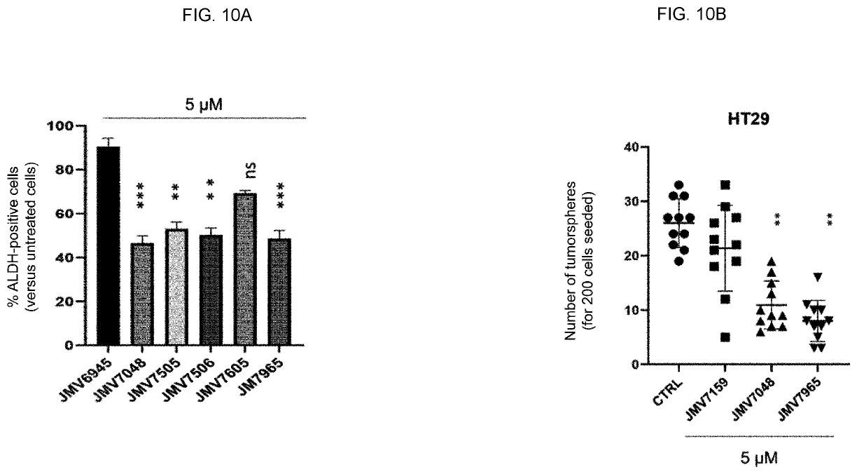

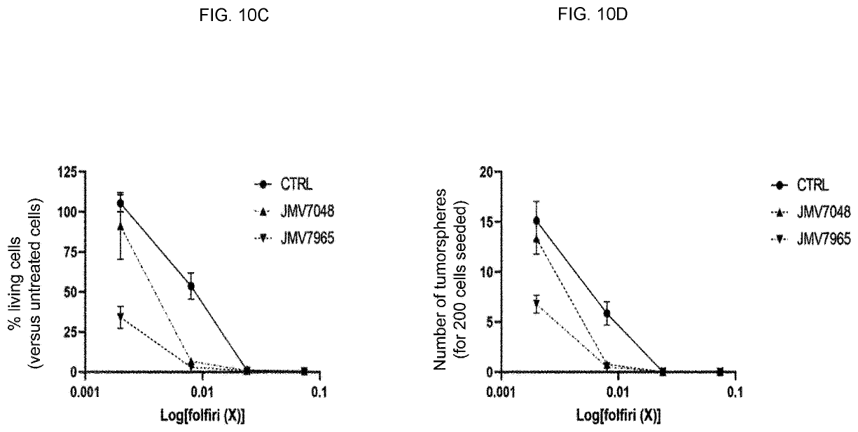

The effects of the PROTACs on the survival and self-renewal of colic cancer stem cells were studied in vitro on the HT29 line or cancer cells isolated from patients (CRC1). The cells were treated with or without 5 μM PROTACs for 48 h before being analyzed: Aldefluor marking, enzymatic activity preferentially present in cancer stem cells (FIG. 10A); formation of tumorspheres under aseric and non-adherent conditions (FIG. 10B), and finally resistance to chemotherapy (FIGS. 100 and 10D).

FIG. 10A shows that the PROTACs JMV7048, JMV7505, JMV7506 and JMV7965 significantly reduce the percentage of ALDH-positive cells after dissociation of the CRC1 cells and marking by Aldefluor™ (STEMCELL Technologies) compared to untreated cells. FIG. 10B shows that the molecules JMV7048 and JMV7965 significantly reduce the number of HT29 cells capable of surviving anoikis and of inducing the formation of tumorspheres (Sphere Forming Cells). The tumorspheres with a diameter of more than 50 μM were counted 10 days after treatment and culturing of 200 cells per well (previously treated with poly2Hema in order to prevent any cell adhesion) in 100 μL of depleted BCS medium. These culture conditions allow only the survival of cancer stem cells. FIGS. 10C and 10D show the effects of the PROTACs JMV7048 and JMV7965 on survival (10C) and the ability to form tumorspheres (10D) of the HT29 cells maintained in the presence of different concentrations of 5-FU and SN38 (Folfiri 1X=50 μg 5-FU+500 nM SN38) and cultured in 100 μL of depleted BCS medium in dishes pretreated with poly2Hema in order to prevent any cell adhesion. The tumorspheres with a diameter of more than 50 μM were counted 10 days after seeding 200 cells/well. Thus, FIGS. 10A-D show that a treatment at 5 μM for 2 days with the PROTACs JMV7048 and JMV7965 significantly reduces the survival and chemoresistance of stem cells in colon cancer cell lines.

1. A bifunctional compound having the general formula (I):
L(PXR)-Linker-L(E3 ligase) (I)
wherein:
L(PXR) is a ligand capable of binding to the PXR nuclear receptor,
L(E3 ligase) represents a ligand of the E3-ubiquitin ligase, and
Linker represents a group which makes it possible to covalently bond L(PXR) to L(E3 ligase).

2. The bifunctional compound according to claim 1, wherein L(PXR) is a group of formula (II): 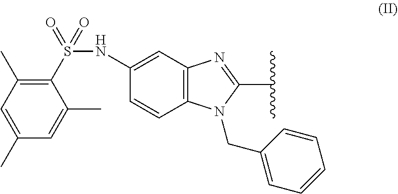 [SMILES: *c1nc2cc(NS(=O)(=O)c3c(C)cc(C)cc3C)ccc2n1Cc1ccccc1]
wherein 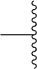
represents the attachment of the group to Linker;
or a pharmaceutically acceptable salt.

3. The compound according to claim 1, wherein L(E3 ligase) is selected from:
the groups of formula (IIIA): 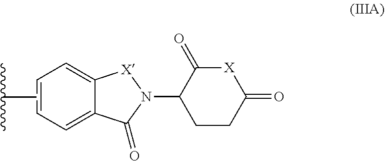
and
the groups of formula (IIIB): 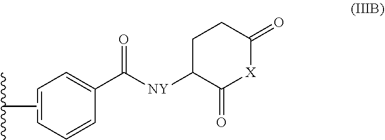
or a pharmaceutically acceptable salt,
wherein:
X is NH;
X is —C(O)— or —CH₂—;
Y represents H or a C1-C6 alkyl group; 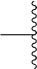
represents the attachment of the group to Linker.

4. The compound of claim 1, wherein Linker represents a C1-C20 alkylene group, optionally interrupted or optionally terminating at either and/or both ends, by one of the groups —O—, —S—, —N(R′)—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —S(O)₂—, —OS(O)—, —S(O)O—, —S(O)—, —OS(O)₂—, —N(R′)S(O)₂—, —S(O)₂N(R′)—, —N(R′)S—, —S(O)N(R′)—, —N(R′)S(O)₂N(R′)—, —N(R′)S(O)N(R′)—, C₃-C₁₂ carbocyclene, 3-to-12-membered heterocyclene comprising 1, 2 or 3 heteroatoms selected from N, O, S, 5-to-12-membered heteroarylene comprising 1, 2 or 3 heteroatoms selected from N, O, S, or any combination thereof, and wherein R′, identical or different, represent H or a C₁-C₆ alkyl group.

5. The compound according to claim 1, wherein Linker represents a group (IV): 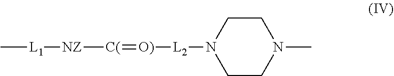
wherein L₁ and L₂, identical or different, independently represent an alkylene group of 1 to 12 carbon atoms optionally interrupted or terminating by a 3-to-12-membered heterocyclene comprising 1, 2 or 3 heteroatoms selected from N, O, S;
L₁ is linked to L(PXR) and 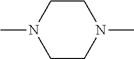
is linked to L(E3 ligase);
Z represents H or a C1-C6 alkyl group.

6. The compound according to claim 1, wherein it conforms to one of the following formulas (I-4) and (I-5): 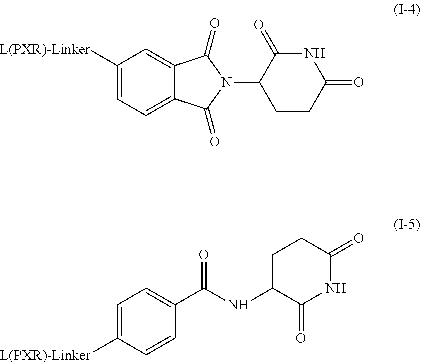
wherein L(PXR), Linker are defined according to claim 1;
or a pharmaceutically acceptable salt.

7. The compound according to claim 1, such that it conforms to the following formula (V): 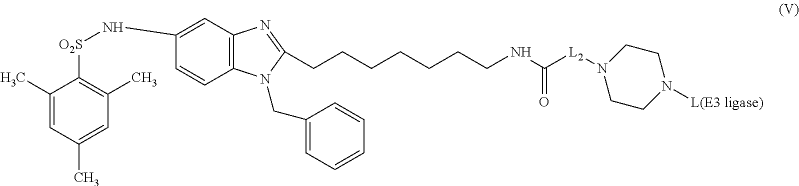
wherein L₂ represents a C2-C8 linear alkylene group optionally interrupted by a piperidinyl group, and L(E3 ligase) is as defined according to claim 1.

8. The compound according to claim 1, wherein it conforms to one of the following formulas:

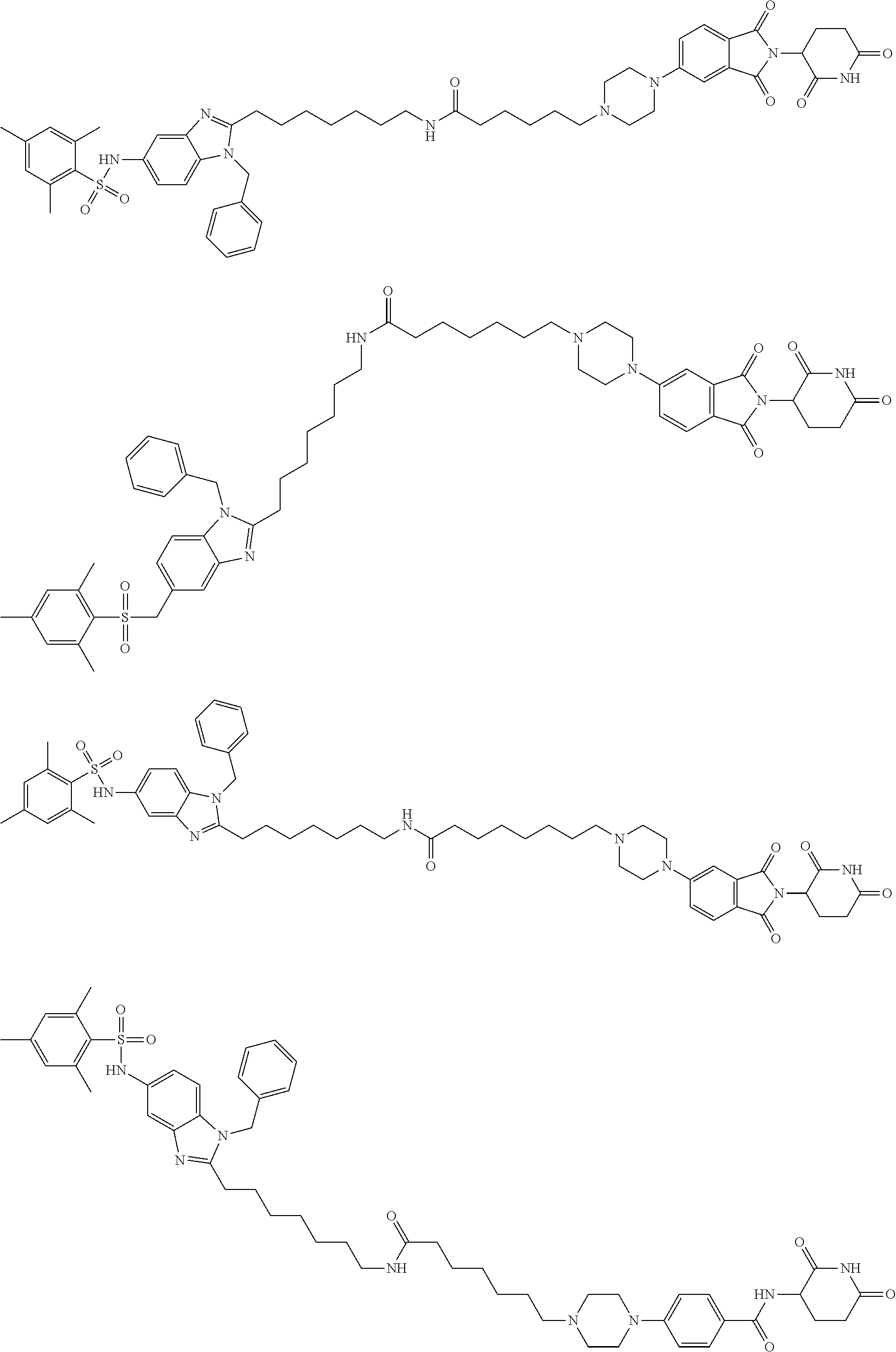 [SMILES: Cc1cc(C)c(S(=O)(=O)Nc2ccc3c(c2)nc(CCCCCCCNC(=O)CCCCCN2CCN(c4ccc5c(c4)C(=O)N(C4CCC(=O)NC4=O)C5=O)CC2)n3Cc2ccccc2)c(C)c1 ; Cc1cc(C)c(S(=O)(=O)Cc2ccc3c(c2)nc(CCCCCCCNC(=O)CCCCCCN2CCN(c4ccc5c(c4)C(=O)N(C4CCC(=O)NC4=O)C5=O)CC2)n3Cc2ccccc2)c(C)c1 ; Cc1cc(C)c(S(=O)(=O)Nc2ccc3c(c2)nc(CCCCCCCNC(=O)CCCCCCCN2CCN(c4ccc5c(c4)C(=O)N(C4CCC(=O)NC4=O)C5=O)CC2)n3Cc2ccccc2)c(C)c1 ; Cc1cc(C)c(S(=O)(=O)Nc2ccc3c(c2)nc(CCCCCCCNC(=O)CCCCCCN2CCN(c4ccc(C(=O)NC5CCC(=O)NC5=O)cc4)CC2)n3Cc2ccccc2)c(C)c1]

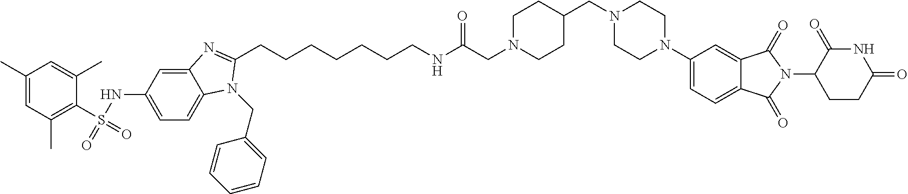 [SMILES: Cc1cc(C)c(S(=O)(=O)Nc2ccc3c(c2)nc(CCCCCCCNC(=O)CN2CCC(CN4CCN(c5ccc6c(c5)C(=O)N(C5CCC(=O)NC5=O)C6=O)CC4)CC2)n3Cc2ccccc2)c(C)c1]

9. A method for preparing a compound according to claim 1, comprising the coupling of a compound of formula (B) and of a compound of formula (C): 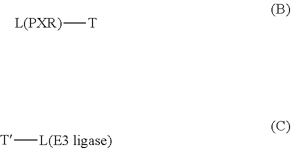
wherein L(PXR) and L(E3 ligase) are as defined according to claim 1, and T and T′ are two groups of Linker precursors, such that they each have respectively a complementary reactive terminal function.

10. The method according to claim 9, wherein the compound (B) conforms to the formula (A): 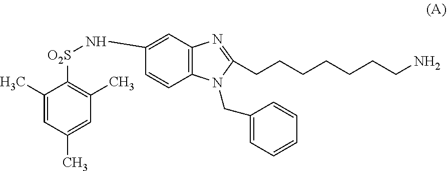 [SMILES: Cc1cc(C)c(S(=O)(=O)Nc2ccc3c(c2)nc(CCCCCCCN)n3Cc2ccccc2)c(C)c1]
and the compound (C) conforms to the formula (C-1): 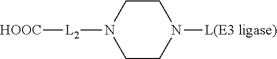
wherein L₂ and L(E3 ligase) are as defined according to claim 1.

11. A compound of formula (A):

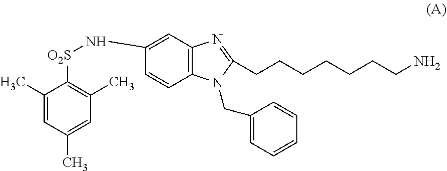 [SMILES: Cc1cc(C)c(S(=O)(=O)Nc2ccc3c(c2)nc(CCCCCCCN)n3Cc2ccccc2)c(C)c1]

12. A pharmaceutical composition comprising a compound according to claim 1, and at least one pharmaceutically acceptable excipient.

13. The compound according to claim 1, for use in the treatment and/or prevention of cancer overexpressing the PXR nuclear receptor.

14. The compound for use according to claim 13 in combination with an anti-cancer agent.

15. The compound for use according to claim 13, for administration to patients who are resistant to anti-cancer agents.
